(19) United States
(12) Patent Application Publication
(10) Pub. No.: US 2010/0050276 A1
(43) Pub. Date: Feb. 25, 2010

(54) Transgenic non-human animal models of apoptosis-mediated conditions

(75) Inventor: Monique E. DePaepe, Barrington, RI (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Brown University, Providence, RI (US)

(21) Appl. No.: 12/229,438

(22) Filed: Aug. 22, 2008

(51) Int. Cl. A01K 67/027; C12N 15/11

(52) U.S. Cl. 800/3; 800/9; 536/23.5; 800/22

(57) ABSTRACT

A transgenic non-human animal whose genome comprises a stable integration of a transgene that encodes at least one Fas-ligand protein operably-linked to a tetracycline-inducible promoter includes cells that express the transgene and undergo apoptosis. The transgenic non-human animal can be used to screen for compounds that inhibit apoptosis and to identify cells that are capable of differentiating in vivo.

### GOVERNMENT SUPPORT

The invention was supported, in whole or in part, by a grant P20-RR18728 from the National Institutes of Health. The Government has certain rights in the invention.

### BACKGROUND OF THE INVENTION

Apoptosis, or programmed cell death, is an integral component of tissue remodeling, such as tissue remodeling that occurs during development. Perturbations in apoptosis, such as excessive apoptosis or ill-timed apoptosis, have been implicated in a variety of diseases and conditions. Currently, the underlying causes and consequences of many diseases and conditions that are consequent to cell death, including programmed cell death, are unknown despite the availability of animal models for these diseases and conditions. Thus, there is a need to develop new, improved and effective models, in particular animal models for use in the study of apoptosis in disease pathology, and to identify therapeutic compounds that inhibit apoptosis to thereby prevent or treat diseases and conditions.

### SUMMARY OF THE INVENTION

The present invention generally relates to transgenic non-human animals and methods of producing transgenic non-human animals; methods of screening for a compound that inhibits apoptosis; and methods of identifying a cell that is capable of differentiating into a target cell employing the transgenic, non-human animals.

In an embodiment, the invention is a transgenic non-human animal whose genome comprises a stable integration of at least one transgene that includes at least one nucleic acid sequence encoding at least one Fas-ligand protein operably-linked to at least one tetracycline-inducible promoter, wherein at least one cell of the transgenic non-human animal that expresses the transgene undergoes apoptosis, and wherein the non-human animal is not a rat.

In another embodiment, the invention is a transgenic non-human animal whose genome comprises a stable integration of at least one first transgene that includes at least one nucleic acid sequence encoding at least one Fas-ligand protein operably-linked to at least one tetracycline-inducible promoter and at least one second transgene that includes at least one second nucleic acid sequence encoding at least one member selected from the group consisting of a reverse tetracycline responsive transactivator protein and a tetracycline responsive transactivator protein.

In a further embodiment, the invention is a recombinant nucleic acid comprising a nucleotide sequence having at least about 75% identity to SEQ ID NO: 4 operably-linked to a tetracycline-inducible promoter, wherein the tetracycline-inducible promoter includes at least seven copies of a tetracycline operator nucleic acid sequence and a cytomegalovirus minimal promoter nucleic acid sequence.

In an additional embodiment, the invention includes a method for producing a transgenic non-human animal, comprising the step of crossing a first transgenic non-human animal whose genome comprises a stable integration of at least one first transgene that includes at least one nucleic acid sequence encoding at least one Fas-ligand protein operably-linked to a tetracycline-inducible promoter with a second transgenic non-human animal whose genome comprises a stable integration of at least one second transgene that includes at least one second nucleic acid sequence encoding at least one reverse tetracycline responsive transactivator protein.

In another embodiment, the invention is a method of screening for a compound that inhibits Fas-ligand mediated apoptosis, comprising the step of assessing Fas-ligand mediated apoptosis in a transgenic non-human animal whose genome comprises a stable integration of at least one first transgene that includes at least one nucleic acid sequence encoding at least one Fas-ligand protein operably-linked to at least one tetracycline-inducible promoter and at least one second transgene that includes at least one second nucleic acid sequence encoding at least one reverse tetracycline responsive transactivator protein, wherein the Fas-ligand mediated apoptosis is in response to administration of the compound in combination with at least one member selected from the group consisting of a tetracycline and a tetracycline analog to the transgenic non-human animal.

In a further embodiment, the invention is a method of identifying a cell that is capable of differentiating into a target cell, comprising the steps of inducing apoptosis of a population of target cells in a transgenic non-human animal whose genome comprises a stable integration of at least one first transgene that includes at least one nucleic acid sequence encoding at least one Fas-ligand protein operably-linked to at least one tetracycline-inducible promoter and at least one second transgene that includes a second nucleic acid sequence encoding at least one member selected from the group consisting of a reverse tetracycline responsive transactivator protein and a tetracycline responsive transactivator protein, and wherein the first transgene and the second transgene are co-expressed in the target cells; introducing at least one cell into the transgenic non-human animal, wherein the cell is selected form the group consisting of a stem cell, a progenitor cell and a bone marrow-derived cell; and detecting differentiation of the cell into a phenotype characteristic of the target cell.

The transgenic non-human animals of the invention can be employed to study the role of apoptosis in various diseases and to identify compounds that inhibit apoptosis. Advantages of the claimed invention include, for example, improved methods of screening for compounds having therapeutic utility in the treatment of apoptosis-mediated conditions.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 depicts constructs used to generate tetracycline-inducible FasL expression in the lung. A: Activator line: the CCSP-rtTA transgene consists of the 2.3-kb rat (r) Clara cell secretory protein (CCSP) promoter, 1.0-kb rtTA coding sequence, and 2.0-kb intronic and polyadenylation sequences from the human growth hormone (hGH)0.38 B: Responder line: the (TetOp)7-FasL transgene consists of seven copies of the tet operator, flanked by a CMV minimal promoter. The 943-bp murine FasL coding sequence was subcloned downstream from the CMV minimal promoter.

FIG. 2 depicts real-time quantitative PCR analysis of FasL mRNA transcripts. A: Analysis of FasL mRNA transcripts in lung homogenates of Dox-treated or non-Dox-treated transgenic mice at P7. *P<0.01 versus Dox-treated CCSP⁺/FasL⁻ littermates. B: Analysis of FasL mRNA transcripts in alveolar type II cell lysates from Dox-treated transgenic mice at E19. *P<0.01 versus Dox-treated CCSP⁺/FasL⁻ littermates. C: Analysis of FasL mRNA expression in various organs of Dox-treated CCSP⁺/FasL mice at P7.

FIG. 3 depicts immunohistochemical analysis of FasL protein expression. A: Representative FasL immunostaining of lungs of Dox-treated CCSP⁺/FasL⁻ mice at E19 showing diffuse and intense FasL immunoreactivity, localized to alveolar epithelial cells morphologically consistent with type II cells, intra-alveolar cellular debris (arrows), and bronchial epithelial cells. B: Lungs of single-transgenic CCSP⁺/FasL⁻ littermates showing modest FasL immunolabeling in bronchial epithelial cells and scattered alveolar epithelial type II cells (arrows). C: Negative control (omission of primary antibody). Anti-FasL immunostaining by ABC method, hematoxylin counterstain. Original magnifications, ×400.

FIG. 4 depicts morphology and TUNEL labeling of lungs of Dox-treated transgenic mice at E19 and P7. A: CCSP⁺/FasL⁺ lungs at E19 (late gestation, saccular stage of development) showing abundant cellular debris within the airspaces and pyknotic nuclei within the bronchial epithelium. Br, bronchus. B: Lungs of CCSP⁺/FasL⁻ littermate of A without histopathological evidence of apoptosis. C: TUNEL labeling of Dox-treated CCSP⁺/Fas⁺ lungs at E19 showing abundant aggregates of TUNEL-positive nuclear material within the alveolar septa and airspaces. In addition, TUNEL-positive nuclei are noted within the bronchial epithelium. Br, bronchus. D: Lungs of a CCSP⁺/FasL⁻ littermate containing only rare TUNEL-positive nuclei, localized to peribronchial and perivascular stromal cells. E: CCSP⁺/FasL⁺ lungs at P7 showing relatively large airspaces, little evidence of secondary crest formation, and abundant intra-alveolar macrophages admixed with apoptotic nuclear debris. Septa appear relatively hypercellular. F: Lungs of CCSP⁺/FasL⁻ littermate of C showing thinner septa, more advanced secondary crest formation (early alveolar stage), and only scant intra-alveolar macrophages. G: CCSP⁺/FasL⁺ lungs at P7 showing persistently high TUNEL positivity, mainly within intra-alveolar cellular debris. H: Lungs of CCSP⁺/FasL⁻ littermate at P7 showing very low TUNEL reactivity. I: Apoptotic index. Values represent mean±SD of at least four animals per group. *P<0.001 versus Dox-treated CCSP⁺/FasL⁻ littermates. H&E staining (A, B, E, F); TUNEL-FITC labeling (C, D, G, H). Original magnifications, X400.

FIG. 5 depicts TUNEL labeling combined with anti-prosurfactant protein C (SP-C) or anti-Clara cell secretory protein (CCSP) immunohistochemistry. A-D: Lungs of Dox-treated transgenic mice at E19. A: Dox-treated CCSP⁺/FasL⁺ lungs at E19 showing co-localization of SP-C-positive cellular material with TUNEL-positive nuclei in intra-alveolar cellular debris (arrows). B: Lungs of littermate of A showing low apoptotic activity in SP-C-negative, non-type II cell. Nonapoptotic SP-C-positive type II cells are noted along the alveolar walls. C: Dox-treated CCSP⁺/FasL⁺ lungs at E19 showing numerous TUNEL-positive nuclei in CCSP-positive bronchial epithelial (Clara) cells. D: Lung of littermate of animal in E showing rare TUNEL positivity, localized to CCSP-negative stromal cells. E-H: Lungs of Dox-treated transgenic mice at P7. E: Lungs of Dox-treated CCSP⁺/FasL⁺ mouse at P7 showing abundant SP-C-negative apoptotic material within the alveoli and within the bronchial lining. Large numbers of intensely SP-C-positive, TUNEL-negative type II cells are noted within and along the alveolar septa. F: Single-transgenic CCSP⁺/FasL⁺ lungs at P7 showing only scattered TUNEL positivity, mainly in SP-C-negative stromal cells. G: CCSP⁺/FasL⁺ lung at P7 showing TUNEL-positive nuclei in and around bronchial epithelial cells. H: Single-transgenic CCSP⁺/FasL⁺ lung at P7 showing occasional apoptotic activity in CCSP-negative cells. br, bronchus. TUNEL: FITC labeling (green), anti-SP-C and anti-CCSP: Cy-3 labeling (red). Original magnifications, X400.

FIG. 6 depicts electron microscopic analysis of lungs of Dox-treated transgenic mice. A: Representative electron micrograph of the alveolar wall of Dox treated single-transgenic CCSP⁺/FasL⁻ mice (E19). A well preserved alveolar type II cell is noted, characterized by lamellar bodies, subnuclear glycogen pools, and abundant microvilli. Myelin whorls, representing surfactant lipids, are present within the airspace (arrow). B: Higher magnification of alveolar type II cell in A showing typical surfactant-containing lamellar bodies (arrows). C: Representative electron micrograph of lungs of Dox-treated double-transgenic CCSP⁺/FasL⁺ mice showing several cells detached from the alveolar wall. The nuclei show characteristic ultrastructural features of apoptosis, including fragmentation, pyknosis, and chromatin condensation. One dying cell contains residual lamellar bodies. D: Higher magnification of cells in C showing the presence of cytoplasmic lamellar bodies (arrows), identifying the dying cells as alveolar type II cells. E: Lung of Dox-treated CCSP⁺/FasL⁺ mouse showing numerous apoptotic nuclei within the airspaces, associated with pools of degenerating myelin-like material. Endothelial cells (end) appear viable. F: Bronchial epithelium of Dox-treated CCSP⁺/FasL⁺ mouse showing a row of apoptotic cells with typical peripheral condensation of chromatin (arrows). An apoptotic nucleus, presumably phagocytosed, is seen in the cytoplasm of a non-Clara bronchial epithelial cell (asterisk).

FIG. 7 depicts Western blot analysis of caspase-3 cleavage. A: Western blot analysis of caspase processing in lung lysates of Dox-treated CCSP⁺/FasL⁺ and CCSP⁺/FasL⁻ mice at P7. In double-transgenic CCSP⁺/FasL⁺ mice, Dox administration resulted in increased levels of the 17- and 20-kDa subunits of caspase-3. Cleavage products were negligible in Dox-treated single-transgenic CCSP⁺/FasL⁻ littermates and non-Dox-treated animals. B: Densitometry of caspase cleavage by Western blot analysis. Values represent combined integrated optical density of 17-kDa and 20-kDa bands as mean±SD. At least four animals were studied per group. *P<0.01 versus Dox-treated CCSP⁺/FasL⁻.

FIG. 8 depicts gross and microscopic appearance of lungs of Dox-treated transgenic mice at P21. A and B: Macroscopic appearance of formalin-inflated lungs of Dox-treated CCSP⁺/FasL⁺ and CCSP⁺/FasL⁻ mice at P21. Lungs of double-transgenic pups appeared large and pale with obtunded edges. C and D: Representative photomicrographs of lungs of Dox-treated CCSP⁺/FasL⁺ and CCSP⁺/FasL⁻ mice at P21. C: CCSP⁺/FasL⁺ lungs at P21 showing large-sized simple alveolar spaces separated by thin alveolar septa. Small macrophage collections are present. D: Lungs of CCSP⁺/FasL⁻ littermate of C showing a complex network of small-sized alveoli (advanced alveolar stage). H&E staining. Original magnifications, ×400.

FIG. 9 depicts MCL and RAC of lungs of Dox-treated or non-Dox-treated transgenic mice at P21. Values are mean±SD of at least six animals studied per group. *P<0.01 versus Dox-treated CCSP⁺/FasL⁻ littermates; §P<0.01 versus-Dox-treated animals of same genotype (Student's t-test).

FIG. 10 depicts pulmonary vascular density of Dox-treated transgenic mice at P21. Representative Factor VIII (von Willebrand factor) immunostaining of lungs of Dox-treated double- and single-transgenic littermates. The density of vessels, highlighted by staining for Factor VIII, is lower in lungs of double transgenic CCSP⁺/FasL⁺ mice (A) compared with CCSP⁺/FasL⁻ littermates (B). C: Vascular density, expressed as number of vessels (20 to 80 μm) per X20 high-power field. Values are mean±SD of four animals studied per group. *P<0.05 versus Dox-treated CCSP⁺/FasL⁻ littermates (Student's t-test).

### DETAILED DESCRIPTION OF THE INVENTION

The features and other details of the invention, either as steps of the invention or as combinations of parts of the invention, will now be more particularly described and pointed out in the claims. It will be understood that the particular embodiments of the invention are shown by way of illustration and not as limitations of the invention. The principal features of this invention can be employed in various embodiments without departing from the scope of the invention.

In an embodiment, the invention is a transgenic non-human animal whose genome comprises a stable integration of at least one transgene that includes at least one nucleic acid sequence encoding at least one Fas-ligand (Fas-L) protein operably-linked to at least one tetracycline-inducible promoter, wherein at least one cell of the transgenic non-human animal that expresses the transgene undergoes apoptosis.

The transgenic non-human animal can be a non-human mammal, provided that the mammal is not a rat. Exemplary non-human mammals include mice, guinea pigs, hamsters, rabbits, goats, sheep, cattle, and pigs. Methods of generating transgenic animals are known to one of skill in the art (see, for example, Pinkert, C. A., Transgenic Animal Technology, Second Edition: A Laboratory Handbook (2002), Elsevier Science, USA).

In an embodiment, the non-human animal is a mouse.

In an embodiment, the cell of the transgenic non-human animal that expresses the transgene and undergoes apoptosis can be a somatic cell (e.g., an epithelial cell or non-epithelial cell, such as a muscle cell, a nerve cell or a connective tissue cell). In another embodiment, the cell of the transgenic non-human animal that expresses the transgene and undergoes apoptosis can be a germ cell.

In an embodiment, the transgenic non-human animal is fertile (e.g., capable of reproducing). In a further embodiment, the transgenic non-human animal survives (e.g., does not die) when apoptosis is induced.

The transgenic non-human animal has a genome that includes the stable integration of between about two to about thirty-five (e.g., about twenty) copies of the transgene. For example, the transgenic non-human animal can have a genome that includes the stable integration of about 5, about 10, about 15, about 20, about 25, about 30 or about 35 copies of the transgene.

In an embodiment, the transgenic non-human animal has between about a 10-fold to about a 200-fold (e.g., about a 30-fold) increase in Fas-ligand mRNA levels compared to a control cell. For example, the transgenic non-human animal can have about a 10-fold, about a 20-fold, about a 30-fold, about a 40-fold, about a 50-fold, about a 100-fold, about a 150-fold or about a 200-fold increase in Fas-ligand mRNA levels compared to a control cell.

“Control cell,” as used herein, refers to a cell that includes a reference level of Fas-ligand mRNA. For example, a control cell can be a cell (e.g., an isolated cell) from another animal, such as an animal whose genome does not include the transgene encoding a Fas-ligand protein (e.g., a transgenic animal having the genotype rtTA⁺/(tetOp)₇-FasL⁻); an animal whose genome includes the transgene encoding a Fas-ligand protein, wherein the animal has not been exposed to tetracycline or a tetracycline analog; an animal whose genome includes the transgene encoding a Fas-ligand protein, but does not include a gene encoding a reverse tetracycline responsive transactivator protein or a tetracycline responsive transactivator protein (e.g., an animal having the genotype tTA⁻/rtTA⁻/(tetOp)₇-FasL⁺). Alternatively, or additionally, the control cell can be a cell (e.g., an isolated cell) that does not express the transgene from the same non-human transgenic animal, or a cultured cell (e.g., a tissue culture cell).

Methods of determining Fas-ligand mRNA levels in a biological sample are well known in the art and include, for example, Northern blotting and real-time quantitative reverse transcriptase polymerase chain reaction (qRT-PCR) (see, e.g., Sambrook, Fritsch & Maniatis, Molecular Cloning: A Laboratory Manual, Second Edition (1989) Cold Spring Harbor Laboratory Press, Cold Spring Harbor, N.Y.; F. M. Ausubel et al. (eds.), Current Protocols in Molecular Biology, John Wiley & Sons, Inc. (1994)).

The transgene can include a nucleic acid sequence encoding at least one mammalian (e.g., murine, rat, human) Fas-ligand protein. Exemplary Fas-ligand protein sequences encoding mammalian Fas-ligand proteins include the following:

FasL protein (mouse): GenPept Accession no. P41047

(SEQ ID NO: 1)

MQQPMNYPCPQIFWVDSSATSSWAPPGSVFPCPSCGPRGPDQRRPPPPPP
PVSPLPPPSQPLPLPPLTPLKKKDHNTNLWLPVVFFMVLVALVGMGLGMY
QLFHLQKELAELREFTNQSLKVSSFEKQIANPSTPSEKKEPRSVAHLTGN
PHSRSIPLEWEDTYGTALISGVKYKKGGLVINETGLYFVYSKVYFRGQSC
NNQPLNHKVYRNSKYPEDLVLMEEKRLNYCTTGQIWAHSSYLGAVFNLTS
ADHLYVNISQLSLINFEESKTFFGLYKL

FasL protein (rat): GenPept Accession no. P36940

(SEQ ID NO: 2)

MQQPVNYPCPQIYWVDSSATSPWAPPGSVFSCPSSGPRGPGQRRPPPPPP
PPSPLPPPSQPPPLPPLSPLKKKDNIELWLPVIFFMVLVALVGMGLGMYQ
LFHLQKELAELREFTNHSLRVSSFEKQIANPSTPSETKKPRSVAHLTGNP
RSRSIPLEWEDTYGTALISGVKYKKGGLVINEAGLYFVYSKVYFRGQSCN
SQPLSHKVYMRNFKYPGDLVLMEEKKLNYCTTGQIWAHSSYLGAVFNLTV
ADHLYVNISQLSLINFEESKTFFGLYKL

FasL protein (human): GenPept Accession no. P48023

(SEQ ID NO: 3)

MQQPFNYPYPQIYWVDSSASSPWAPPGTVLPCPTSVPRRPGQRRPPPPPP
PPPLPPPPPPPPLPPLPLPPLKKRGNHSTGLCLLVMFFMVLVALVGLGLG
MFQLFHLQKELAELRESTSQMHTASSLEKQIGHPSPPPEKKELRKVAHLT
GKSNSRSMPLEWEDTYGIVLLSGVKYKKGGLVINETGLYFVYSKVYFRGQ
SCNNLPLSHKVYMRNSKYPQDLVMMEGKMMSYCTTGQMWARSSYLGAVFN
LTSADHLYVNVSELSLVNFEESQTFFGLYKL

TABLE 1

CLUSTAL 2.0.8 multiple sequence alignment of mouse, rat and human
FasL amino acid sequences (SEQ ID NOS: 1-3)

| | | |
|---|---|---|
| mouse | MQQPMNYPCPQIFWVDSSATSSWAPPGSVFPVPSCGPRGPDQRRPPPPPPPVSPLPPPSQ | 60 |
| Rat | MQQPVNYPCPQIYWVDSSATSPWAPPGSVFSCPSSGPRGPGQRRPPPPPPPPSPLPPPSQ | 60 |
| Human | MQQPFNYPYPQIYWVDSSASSPWAPPGTVLPCPTSVPRRPGQRRPPPPPPPP-PLPPPPP | 59 |
| | ****.*** ***:******:*.*****:*:.**:. ** *.********** *****. | |
| mouse | PLPLP--PLTPLKKKDH-NTNLWLPVVFFMVLVALVGMGLGMYQLFHLQKELAELREFTN | 117 |
| Rat | PPPLP--PLSPLKKKD--NIELWLPVIFFMVLVALVGMGLGMYQLFHLQKELAELREFTN | 116 |
| Human | PPPLPPLPLPPLKKRGNHSTGLCLLVMFFMVLVALVGLGLGMFQLFHLQKELAELRESTS | 119 |
| | * *** **.****:.**. * * *:**********:****:************** *. | |
| mouse | QSLKVSSFEDQIANPSTPSEKKEPRSVAHLTGNPHSRSIPLEWEDTYGTALISGVKYKKG | 177 |
| Rat | HSLRVSSFEKQIANPSTPSETKKPRSVAHLTGNPRSRSIPLEWEDTYGTALISGVKYKKG | 176 |
| Human | QMHTASSLEKQIGHPSPPPEKKELRKVAHLTGKSNSRSMPLEWEDTYGIVLLSGVKYKKG | 179 |
| | : .**:****.:**.*.*.*: *.******:..***:********* .*:******** | |
| mouse | GLVINETGLYFVYSKVYFRGQSCNNQPLNHKVYMRNSKYPEDLVLMEEKRLNYCTTGQIW | 237 |
| Rat | GLVINEAGLYFVYSKVYFRGQSCNSQPLSHKVYMRNFKYPGDLVLMEEKKLNYCTTGQIW | 236 |
| Human | GLVINETGLYFVYSKVYFRGQSCNNLPLSHKVYMRNSKYPQDLVMMEGKMMSYCTTGQMW | 239 |
| | ******:*****************.-**.******* *** ***:** * :.******:* | |
| mouse | AHSSYLGAVFNLTSADHLYVNISQLSLINFEESKTFFGLYKL | 279 |
| Rat | AHSSYLGAVFNLTVADHLYVNISQLSLINFEESKTFFGLYKL | 287 |
| Human | ARSSYLGAVFNLTSADHLYVNVSELSLVNFEESQTFFGLYKL | 281 |
| | *:*********** *******:*:***:*****:******** | |

*-residues or nucleotides in that column are identical in all sequences in the alignment
:-conserved substitutions have been observed
.-semi-conserved substitutions have been observed
Homology mouse-rat amino acid sequence: 91%
Homology mouse-human amino acid sequence: 75%

Exemplary cDNA sequences encoding mammalian Fas-ligand proteins include the following:

FasL cDNA(mouse): GenBank Accession no. NM_{—}010177

(SEQ ID NO: 4)

TGAGGCTTCTCAGCTTCAGATGCAAGTGAGTGGGTGTCTCACAGAGAAGC
AAAGAGAAGAGAACAGGAGAAAGGTGTTTCCCTTGACTGCGGAAACTTTA
TAAAGAAAACTTAGCTTCTCTGGAGCAGTCAGCGTCAGAGTTCTGTCCTT
GACACCTGAGTCTCCTCCACAAGGCTGTGAGAAGGAAACCCTTTCCTGGG
GCTGGGTGCCATGCAGCAGCCCATGAATTACCCATGTCCCCAGATCTTCT
GGGTAGACAGCAGTGCCACTTCATCTTGGGCTCCTCCAGGGTCAGTTTTT
CCCTGTCCATCTTGTGGGCCTAGAGGGCCGGACCAAAGGAGACCGCCACC
TCCACCACCACCTGTGTCACCACTACCACCGCCATCACAACCACTCCCAC
TGCCGCCACTGACCCCTCTAAAGAAGAAGGACCACAACACAAATCTGTGG
CTACCGGTGGTATTTTTCATGGTTCTGGTGGCTCTGGTTGGAATGGGATT
AGGAATGTATCAGCTCTTCCACCTGCAGAAGGAACTGGCAGAACTCCGTG
AGTTCACCAACCAAAGCCTTAAAGTATCATCTTTTGAAAAGCAAATAGCC
AACCCCAGTACACCCTCTGAAAAAAAAGAGCCGAGGAGTGTGGCCCATTT
AACAGGGAACCCCCACTCAAGGTCCATCCCTCTGGAATGGGAAGACACAT
ATGGAACCGCTCTGATCTCTGGAGTGAAGTATAAGAAAGGTGGCCTTGTG
ATCAACGAAACTGGGTTGTACTTCGTGTATTCCAAAGTATACTTCCGGGG
TCAGTCTTGCAACAACCAGCCCCTAAACCACAAGGTCTATATGAGGAACT
CTAAGTATCCTGAGGATCTGGTGCTAATGGAGGAGAAGAGGTTGAACTAC
TGCACTACTGGACAGATATGGGCCCACAGCAGCTACCTGGGGGCAGTATT
CAATCTTACCAGTGCTGACCATTTATATGTCAACATATCTCAACTCTCTC
TGATCAATTTTGAGGAATCTAAGACCTTTTTCGGCTTGTATAAGCTTTAA
AAGAAAAAGCATTTTAAAATGATCTACTATTCTTTATCATGGGCACCAGG
AATATTGTCTTGAATGAGAGTCTTCTTAAGACCTATTGAGATTAATTAAG
ACTACATGAGCCACAAAGACCTCATGACCGCAAGGTCCAACAGGTCAGCT
ATCCTTCATTTTCTCGAGGTCCATGGAGTGGTCCTTAATGCCTGCATCAT
GAGCCAGATGGAAGGAGGTCTGTGACTGAGGGACATAAAGCTTTGGGCTG
CTGTGTGACAATGCAGAGGCACAGAGAAAGAACTGTCTGATGTTAAATGG
CCAAGAGAATTTTAACCATTGAAGAAGACACCTTTACACTCACTTCCAGG
GTGGGTCTACTTACTACCTCACAGAGGCCGTTTTTGAGACATAGTTGTGG
TATGAATATACAAGGGTGAGAAAGGAGGCTCATTTGACTGATAAGCTAGA
GACTGAAAAAAAGACAGTGTCTCATTGGCACCATCTTTACTGTTACCTAA
TGTTTTCTGAGCCGACCTTTGATCCTAACGGAGAAGTAAGAGGGATGTTT
GAGGCACAAATCATTCTCTACATAGCATGCATACCTCCAGTGCAATGATG
TCTGTGTGTTTGTATGTATGAGAGCAAACAGATTCTAAGGAGTCATATAA
ATAAAATATGTACATTATGGAGTACATATTAGAAACCTGTTACATTTGAT
GCTAGATATCTGAATGTTTCTTGGCAATAAACTCTAATAGTCTTCAAAAT
CTTTTATTATCAGCTACTGATGCTGTTTTTCTTTAATACAACTAGTATTT
ATGCTCTGAACATCCTAATGAGGAAAAGACAAATAAAATTATGTTATAGA
ATACAGAAATGCCTTAAGGACATAGACTTTGGAAATC

FasL cDNA(rat): GenBank Accession No. NM_{—}012908

(SEQ ID NO: 5)

TCAGAGTCCTGTCCTTGACACTTCAGTCTCCACAAGACTGAGAGGAGGAA
ACCCTTTCCTGGGGCTGGGTGCCATGCAGCAGCCCGTGAATTACCCATGT
CCCCAGATCTACTGGGTAGACAGCAGTGCCACTTCTCCTTGGGCTCCTCC
AGGGTCAGTTTTTTCTTGTCCATCCTCTGGGCCTAGAGGGCCAGGACAAA
GGAGACCACCGCCTCCACCACCACCTCCATCACCACTACCACCGCCTTCC
CAACCACCCCCGCTGCCTCCACTAAGCCCTCTAAAGAAGAAGGACAACAT
AGAGCTGTGGCTACCGGTGATATTTTTCATGGTGCTGGTGGCTCTGGTTG
GAATGGGGTTAGGAATGTATCAACTCTTTCATCTACAGAAGGAACTGGCA
GAACTCCGTGAGTTCACCAACCACAGCCTTAGAGTATCATCTTTTGAAAA
GCAAATAGCCAACCCCAGCACACCCTCTGAAACCAAAAAGCCAAGGAGTG
TGGCCCACTTAACAGGGAACCCCCGCTCAAGGTCCATCCCTCTGGAATGG
GAAGACACATATGGAACTGCTTTGATCTCTGGAGTGAAGTATAAGAAAGG
CGGCCTTGTGATCAATGAGGCTGGGTTGTACTTCGTATATTCCAAAGTAT
ACTTCCGGGGTCAGTCTTGCAACAGCCAGCCCCTAAGCCACAAGGTCTAT
ATGAGGAACTTTAAGTATCCTGGGGATCTGGTGCTAATGGAGGAGAAGAA
GTTGAATTACTGCACTACTGGCCAGATATGGGCCCACAGCAGCTACCTAG
GGGCAGTATTTAATCTTACCGTTGCTGACCATTTATATGTCAACATATCT
CAACTCTCTCTGATCAATTTTGAGGAATCTAAGACCTTTTTTGGCTTATA
TAAGCTTTAAAGGAAAAAGCATTTTAGAATGATCTATTATTCTTTATCAT
GGATGCCAGGAATATTGTCTTCAATGAGAGTCTTCTTAAGACCAATTGAG
CCACAAAGACCACAAGGTCCAACAGGTCAGCTACCCTTCATTTTCTAGAG
GTCCATGGAGTGGTCCTTAATGCCTGCATCATGAGCCAGATGGGAAGAAG
ACTGTTCCTGAGGAACATAAAGTTTTGGGCTGCTGTGTGGCAATGCAGAG
GCAAAGAGAAGGAACTGTCTGATGTTAAATGGCCAAGAGCATTTTAGCCA
TTGAAGAAAAAAAAAACCTTTAAACTCACCTTCCAGGGTGGGTCTACTTG
CTACCTCACAGGAGGCCGTCTTTTAGACACATGGTTGTGGTATGACTATA
CAAGGGTGAGAAAGGATGCTAGGTTTCATGGATAAGCTAGAGACTGAAAA
AAGCCAGTGTCCCATTGGCATCATCTTTATTTTTAACTGATGTTTTCTGA
GCCCACCTTTGATGCTAACAGAGAAATAAGAGGGGTGTTTGAGGCACAAG
TCATTCTCTACATAGCATGTGTACCTCCAGTGCAATGATGTCTGTGTGTG
TTTTTATGTATGAGAGTAGAGCGATTCTAAAGAGTCACATGAGTACAACG
CGTACATTACGGAGTACATATTAGAAACGTATGTGTTACATTTGATGCTA
GAATATCTGAATGTTTCTTGCTA

FasL cDNA (human): GenBank Accession No. X89102

(SEQ ID NO: 6)

GAGGTGTTTCCCTTAGCTATGGAAACTCTATAAGAGAGATCCAGCTTGCC
TCCTCTTGAGCAGTCAGCAACAGGGTCCCGTCCTTGACACCTCAGCCTCT
ACAGGACTGAGAAGAAGTAAAACCGTTTGCTGGGGCTGGCCTGACTCACC
AGCTGCCATGCAGCAGCCCTTCAATTACCCATATCCCCAGATCTACTGGG
TGGACAGCAGTGCCAGCTCTCCCTGGGCCCCTCCAGGCACAGTTCTTCCC
TGTCCAACCTCTGTGCCCAGAAGGCCTGGTCAAAGGAGGCCACCACCACC
ACCGCCACCGCCACCACTACCACCTCCGCCGCCGCCGCCACCACTGCCTC
CACTACCGCTGCCACCCCTGAAGAAGAGAGGGAACCACAGCACAGGCCTG
TGTCTCCTTGTGATGTTTTTCATGGTTCTGGTTGCCTTGGTAGGATTGGG
CCTGGGGATGTTTCAGCTCTTCCACCTACAGAAGGAGCTGGCAGAACTCC
GAGAGTCTACCAGCCAGATGCACACAGCATCATCTTTGGAGAAGCAAATA
GGCCACCCCAGTCCACCCCCTGAAAAAAAGGAGCTGAGGAAAGTGGCCCA
TTTAACAGGCAAGTCCAACTCAAGGTCCATGCCTCTGGAATGGGAAGACA
CCTATGGAATTGTCCTGCTTTCTGGAGTGAAGTATAAGAAGGGTGGCCTT
GTGATCAATGAAACTGGGCTGTACTTTGTATATTCCAAAGTATACTTCCG
GGGTCAATCTTGCAACAACCTGCCCCTGAGCCACAAGGTCTACATGAGGA
ACTCTAAGTATCCCCAGGATCTGGTGATGATGGAGGGGAAGATGATGAGC
TACTGCACTACTGGGCAGATGTGGGCCCGCAGCAGCTACCTGGGGGCAGT
GTTCAATCTTACCAGTGCTGATCATTTATATGTCAACGTATCTGAGCTCT
CTCTGGTCAATTTTGAGGAATCTCAGACGTTTTTCGGCTTATATAAGCTC
TAAGAGAAGCACTTTGGGATTCTTTCCATTATGATTCTTTGTTACAGGCA
CCGAGAATGTTGTATTCAGTGAGGGTCTTCTTACATGCATTTGAGGTCAA
GTAAGAAGACATGAACCAAGTGGACCTTGAGACCACAGGGTTCAAAATGT
CTGTAGCTCCTCAACTCACCTAATGTTTATGAGCCAGACAAATGGAGGAA
TATGACGGAAGAACATAGAACTCTGGGCTGCCATGTGAAGAGGGAGAAGC
ATGAAAAAGCAGCTACCAGGTGTTCTACACTCATCTTAGTGCCTGAGAGT
ATTTAGGCAGATTGAAAAGGACACCTTTTAACTCACCTCTCAAGGTGGGC
CTTGCTACCTCAAGGGGGACTGTCTTTCAGATACATGGTTGTGACCTGAG
GATTTAAGGGATGGAAAAGGAAGACTAGAGGCTTGCATAATAAGCTAAAG
AGGCTGAAAGAGGCCAATGCCCCACTGGCAGCATCTTCACTTCTAAATGC
ATATCCTGAGCCATCGGTGAAACTAACAGATAAGCAAGAGAGATGTTTTG
GGGACTCATTTCATTCCTAACACAGCATGTGTATTTCCAGTGCAATTGTA
GGGGTGTGTGTGTGTGTGTGTGTGTGTGTGTGTGTATGACTAAAGAGAGA
ATGTAGATATTGTGAAGTACATATTAGGAAAATATGGGTTGCATTTGGTC
AAGATTTTGAATGCTTCCTGACAATCAACTCTAATAGTGCTTAAAAATCA
TTGATTGTCAGCTACTAATGATGTTTTCCTATAATATAATAAATATTTAT
GTAGATGTGCATTTTTGTGAAATGAAAACATGTAATAAAAAGTATATGTT
AGGATACAAAAAAAAAAAAAAAAAAAAAAAAAAAAAAAAAAAAAAAAAAA
AAAAAAAAA

TABLE 2

CLUSTAL 2.0.8 multiple sequence alignment of mouse, rat and human
FasL cDNA sequences (SEQ ID NOS: 4-6)

| Mouse | GAGAAGG--AAACCCTTTCCTGGGGCTGGG-------------TGCCATGCAGCAGC | 220 |
|---|---|---|
| Rat | GAGGAGG--AAACCCTTTCCTGGGGCTGGG-------------TGCCATGCAGCAGC | 83 |
| Human | GAAGAAGTAAAACCGTTTGCTGGGGCTGGCCTGACTCACCAGCTGCCATGCAGCAGC | 167 |
| | ** * * ***** *** ********** ************** | |
| Mouse | CCATGAATTACCCATGTCCCCAGATCTTCTGGGTAGACAGCAGTGCCACTTCATCTTGGG | 280 |
| Rat | CCGTGAATTACCCATGTCCCCAGATCTACTGGGTAGACAGCAGTGCCACTTCTCCTTGGG | 143 |
| Human | CCTTCAATTACCCATATCCCCAGATCTACTGGGTGGACAGCAGTGCCAGCTCTCCCTGGG | 227 |
| | ** * ********** *********** ****** ************* ** * **** | |
| Mouse | CTCCTCCAGGGTCAGTTTTTCCCTGTCCATCTTGTGGGCCTAGAGGGCCGGACCAAAGGA | 340 |
| Rat | CTCCTCCAGGGTCAGTTTTTTCTTGTCCATCCTCTGGGCCTAGAGGGCCAGGACAAAGGA | 203 |
| Human | CCCCTCCAGGCACAGTTCTTCCCTGTCCAACCTCTGTGCCCAGAAGGCCTGGTCAAAGGA | 287 |
| | * ******** *****-** * ****** * * ** *** *** **** * ******* | |
| Mouse | GACCGCCACCTCCACCACCACCTGTGTCACCACTACC---ACCGCCATCACAACCACTCC | 397 |
| Rat | GACCACCGCCTCCACCACCACCTCCATCACCACTACC---ACCGCCTTCCCAACCACCCC | 260 |
| Human | GGCCACCACCACCACCGCCACCGCCACCACTACCACCTCCGCCGCCGCCGCCACCACTGC | 347 |
| | * ** ** ** ***** ***** *** ** *** ***** * * ***** * | |
| Mouse | CACTGCCGCCACTGACCCCTCTAAAGAAGAAGG---ACCACAACACAAATCTGTGGCTAC | 454 |
| Rat | CGCTGCCTCCACTAAGCCCTCTAAAGAAGAAGG---A---CAACATAGAGCTGTGGCTAC | 314 |
| Human | CTCCACTACCGCTGCCACCCCTGAAGAAGAGAGGGAACCACAGCACAGGCCTGTGTCTCC | 407 |
| | * * * ** ** ** ** ******* * * ** ** * ***** ** * | |
| Mouse | CGGTGGTATTTTTCATGGTTCTGGTGGCTCTGGTTGGAATGGGATTAGGAATGTATCAGC | 514 |
| Rat | CGGTGATATTTTTCATGGTGCTGGTGGCTCTGGTTGGAATGGGGTTAGGAATGTATCAAC | 374 |
| Human | TTGTGATGTTTTTCATGGTTCTGGTTGCCTTGGTAGGATTGGGCCTGGGGATGTTTCAGC | 467 |
| | *** * *********** ***** ** **** *** **** * ** **** *** * | |
| Mouse | TCTTCCACCTGCAGAAGGAACTGGCAGAACTCCGTGAGTTCACCAACCAAAGCCTTAAAG | 574 |
| Rat | TCTTTCATCTACAGAAGGAACTGGCAGAACTCCGTGAGTTCACCAACCACAGCCTTAGAG | 434 |
| Human | TCTTCCACCTACAGAAGGAGCTGGCAGAACTCCGAGAGTCTACCAGCCAGATGCACACAG | 527 |
| | **** ** ** ******** ************** **** **** *** * * * ** | |
| Mouse | TATCATCTTTTGAAAAGCAAATAGCCAACCCCAGTACACCCTCTGAAAAAAAAGAGCCGA | 634 |
| Rat | TATCATCTTTTGAAAAGCAAATAGCCAACCCCAGCACACCCTCTGAAACCAAAAAGCCAA | 494 |
| Human | CATCATCTTTGGAGAAGCAAATAGGCCACCCCAGTCCACCCCCTGAAAAAAAGGAGCTGA | 587 |
| | ********* ** ********** * ******* ***** ****** ** *** * | |
| Mouse | GGAGTGTGGCCCATTTAACAGGGAACCCCCACTCAAGGTCCATCCCTCTGGAATGGGAAG | 694 |
| Rat | GGAGTGTGGCCCACTTAACAGGGAACCCCCGCTCAAGGTCCATCCCTCTGGAATGGGAAG | 554 |
| Human | GGAAAGTGGCCCATTTAACAGGCAAGTCCAACTCAAGGTCCATGCCTCTGGAATGGGAAG | 647 |
| | *** ******** ******** ** ** ************ **************** | |
| Mouse | ACACATATGGAACCGCTCTGATCTCTGGAGTGAAGTATAAGAAAGGTGGCCTTGTGATCA | 754 |
| Rat | ACACATATGGAACTGCTTTGATCTCTGGAGTGAAGTATAAGAAAGGCGGCCTTGTGATCA | 614 |
| Human | ACACCTATGGAATTGTCCTGCTTTCTGGAGTGAAGTATAAGAAGGGTGGCCTTGTGATCA | 707 |
| | **** ******* * ** * ******************** ** ************* | |
| Mouse | ACGAAACTGGGTTGTACTTCGTGTATTCCAAAGTATACTTCCGGGGTCAGTCTTGCAACA | 814 |
| Rat | ATGAGGCTGGGTTGTACTTCGTATATTCCAAAGTATACTTCCGGGGTCAGTCTTGCAACA | 674 |
| Human | ATGAAACTGGGCTGTACTTTGTATATTCCAAAGTATACTTCCGGGGTCAATCTTGCAACA | 767 |
| | * ** ***** ******* ** ************************** ********** | |
| Mouse | ACCAGCCCCTAAACCACAAGGTCTATATGAGGAACTCTAAGTATCCTGAGGATCTGGTGC | 874 |
| Rat | GCCAGCCCCTAAGCCACAAGGTCTATATGAGGAACTTTAAGTATCCTGGGGATCTGGTGC | 734 |
| Human | ACCTGCCCCTGAGCCACAAGGTCTACATGAGGAACTCTAAGTATCCCCAGGATCTGGTGA | 827 |
| | ** ****** * ************ ********** ********* ********** | |
| Mouse | TAATGGAGGAGAAGAGGTTGAACTACTGCACTACTGGACAGATATGGGCCCACAGCAGCT | 934 |
| Rat | TAATGGAGGAGAAGAAGTTGAATTACTGCACTACTGGCCAGATATGGGCCCACAGCAGCT | 794 |
| Human | TGATGGAGGGGAAGATGATGAGCTACTGCACTACTGGGCAGATGTGGGCCCGCAGCAGCT | 887 |
| | * ******* ***** * *** ************** ***** ******* ******** | |
| Mouse | ACCTGGGGGCAGTATTCAATCTTACCAGTGCTGACCATTTATATGTCAACATATCTCAAC | 994 |
| Rat | ACCTAGGGGCAGTATTTAATCTTACCGTTGCTGACCATTTATATGTCAACATATCTCAAC | 854 |
| Human | ACCTGGGGGCAGTGTTCAATCTTACCAGTGCTGATCATTTATATGTCAACGTATCTGAGC | 947 |
| | **** ******** ** ********* ****** *************** ***** * * | |
| Mouse | TCTCTCTGATCAATTTTGAGGAATCTAAGACCTTTTTCGGCTTGTATAAGCTTTAAAAGA | 1054 |
| Rat | TCTCTCTGATCAATTTTGAGGAATCTAAGACCTTTTTTGGCTTATATAAGCTTTAAAGGA | 914 |
| Human | TCTCTCTGGTCAATTTTGAGGAATCTCAGACGTTTTTCGGCTTATATAAGCTCTAAGAGA | 1007 |
| | ******** ***************** **** ***** ***** ******** *** ** | |
| Mouse | AAAAGCATTTTAAAATGATCT-----ACTATTCTTTATCATGGGCACCAGGAATATT | 1109 |
| Rat | AAAAGCATTTTAGAATGATCT-----ATTATTCTTTATCATGGATGCCAGGAATATT | 969 |
| Human | A---GCACTTTGGGATTCTTTCCATTATGATTCTTTGTTACAGGCACCGAGAATGTT | 1064 |
| | * *** *** ** * * * ******* * * * ** **** ** | |
| *-residues or nucleotides in that column are identical in all sequences in the alignment | | |
| :-conserved substitutions have been observed | | |

.-semi-conserved substitutions have been observed
Homology rat cDNA sequences to murine FasL cDNA we employed: 93%.
Homology human cDNA sequences to murine FasL cDNA we employed: 76%.

In an embodiment, the transgene can include a nucleic acid sequence that has at least about 75.0% (e.g., about 76.0%, about 93.0%) identity to a murine Fas-ligand cDNA comprising SEQ ID NO:4. For example, the transgene can include a nucleic acid sequence encoding a Fas-ligand protein has at least about 75.0%, about 80.0%, about 85.0%, about 90.0%, about 95.0% or about 99.0% identity to a murine Fas-ligand cDNA comprising SEQ ID NO:4.

The percent identity of two nucleic acid sequences (or two amino acid sequences) can be determined by aligning the sequences for optimal comparison purposes (e.g., gaps can be introduced in the sequence of a first sequence). The amino acid sequence or nucleic acid sequences at corresponding positions are then compared, and the percent identity between the two sequences is a function of the number of identical positions shared by the sequences (i.e., % identity=# of identical positions/total # of positions×100). The length of the protein or nucleic acid that can be aligned for comparison purposes is at least about 95% of the length of the reference sequence, for example, the murine Fas-ligand cDNA sequence (SEQ ID NO:4) or murine Fas-ligand protein (SEQ ID NO:1).

The actual comparison of the two sequences can be accomplished by well-known methods, for example, using a mathematical algorithm. A preferred, non-limiting example of such a mathematical algorithm is described in Karlin et al. (Proc. Natl. Acad. Sci. USA, 90:5873-5877 (1993)). Such an algorithm is incorporated into the BLASTN and BLASTX programs (version 2.2) as described in Schaffer et al. (Nucleic Acids Res., 29:2994-3005 (2001)). When utilizing BLAST and Gapped BLAST programs, the default parameters of the respective programs (e.g., BLASTN; available at the Internet site for the National Center for Biotechnology Information) can be used. In one embodiment, the database searched is a non-redundant (NR) database, and parameters for sequence comparison can be set at: no filters; Expect value of 10; Word Size of 3; the Matrix is BLOSUM62; and Gap Costs have an Existence of 11 and an Extension of 1.

Another mathematical algorithm employed for the comparison of sequences is the algorithm of Myers and Miller, CABIOS (1989). Such an algorithm is incorporated into the ALIGN program (version 2.0), which is part of the GCG (Accelrys, San Diego, Calif.) sequence alignment software package. When utilizing the ALIGN program for comparing amino acid sequences, a PAM120 weight residue table, a gap length penalty of 12, and a gap penalty of 4 is used. Additional algorithms for sequence analysis are known in the art and include ADVANCE and ADAM as described in Torellis and Robotti (Comput. Appl. Biosci., 10: 3-5 (1994)); and FASTA described in Pearson and Lipman (Proc. Natl. Acad Sci USA, 85: 2444-2448 (1988)).

The percent identity between two amino acid sequences can also be accomplished using the GAP program in the GCG software package (Accelrys, San Diego, Calif.) using either a Blossom 63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the GCG software package (Accelrys, San Diego, Calif.), using a gap weight of 50 and a length weight of 3.

The transgene encoding at least one Fas-ligand protein can be operably-linked to at least one tetracycline-inducible promoter. In an embodiment, the tetracycline-inducible promoter can include at least about seven copies of a tet operator nucleic acid sequence. For example, the tetracycline-inducible promoter can include at least about 7, about 8, about 9, about 10, about 11, about 12, about 13, or about 14 tet operator nucleic acid sequences. In another embodiment, the tetracycline-inducible promoter can include seven copies of the tet operator nucleic acid sequence. An exemplary tet-responsive promoter nucleic acid sequence that includes seven tet operator nucleic acid sequences is SEQ ID NO:7, which is contained in the pTRE2 vector (BD Biosciences, Franklin Lakes, N.J.).

(SEQ ID NO: 7)

CTCGAGTTTACCACTCCCTATCAGTGATAGAGAAAAGTGAAAGTCGAGTT
TACCACTCCCTATCAGTGATAGAGAAAAGTGAAAGTCGAGTTTACCACTC
CCTATCAGTGATAGAGAAAAGTGAAAGTCGAGTTTACCACTCCCTATCAG
TGATAGAGAAAAGTGAAAGTCGAGTTTACCACTCCCTATCAGTGATAGAG
AAAAGTGAAAGTCGAGTTTACCACTCCCTATCAGTGATAGAGAAAAGTGA
AAGTCGAGTTTACCACTCCCTATCAGTGATAGAGAAAAGTGAAAGTCGAG
CTCGGTACCCGGGTCGAGGTAGGCGTGTACGGTGGGAGGCCTATATAAGC
AGAGCTCGTTTAGTGAACCGTCAGATCGCCTGGAGACGCCATCCACGCTG
TTTTGACCTCCATAGAAGACACCGGGACCGATCCAGCC

The tetracycline-inducible promoter can further include a cytomegalovirus minimal promoter nucleic acid sequence. An exemplary a cytomegalovirus minimal promoter nucleic acid sequence is SEQ ID NO:8.

(SEQ ID NO: 8)

TAGGCGTGTACGGTGGGAGGCCTATATAAGCAGAGCTCGTTTAGTGAACC
GTCAGATCGCCTGGAGACGCCATCCACGCTGTTTTGACCTCCATAGAAGA
CACCGGGACCGATCCAGCC.

The transgene encoding at least one Fas-ligand protein can further include a polyadenylation nucleic acid sequence, such as, for example, a beta-globin polyadenylation contained in the pTRE2 vector (BD Biosciences, Franklin Lakes, N.J.):

(SEQ ID NO: 9)

AGCTGAGAACTTCAGGGTGAGTTTGGGGACCCTTGATTGTTCTTTCTTTT
TCGCTATTGTAAAATTCATGTTATATGGAGGGGGCAAAGTTTTCAGGGTG
TTGTTTAGAATGGGAAGATGTCCCTTGTATCACCATGGACCCTCATGATA
ATTTTGTTTCTTTCACTTTCTACTCTGTTGACAACCATTGTCTCCTCTTA
TTTTCTTTTCATTTTCTGTAACTTTTTTCGTTAAACTTTAGCTTGCATTT
GTAACGAATTTTTAAATTCACTTTCGTTTATTTGTCAGATTGTAAGTACT
TTCTCTAATCACTTTTTTTTCAAGGCAATCAGGGTAATTATATTGTACTT
CAGCACAGTTTTAGAGAACAATTGTTATAATTAAATGATAAGGTAGAATA
TTTCTGCATATAAATTCTGGCTGGCGTGGAAATATTCTTATTGGTAGAAA
CAACTACATCCTGGTAATCATCCTGCCTTTCTCTTTATGGTTACAATGAT
ATACACTGTTTGAGATGAGGATAAAATACTCTGAGTCCAAACCGGGCCCC
TCTGCTAACCATGTTCATGCCTTCTTCTTTTTCCTACAGCTCCTGGGCAA
CGTGCTGGTTGTTGTGCTGTCTCATCATTTTGGCAAAGAATTCACTCCTC
AGGTGCAGGCTGCCTATCAGAAGGTGGTGGCTGGTGTGGCCAATGCCCTG
GCTCACAAATACCACTGAGATCTTTTTCCCTCTGCCAAAAATTATGGGGA
CATCATGAAGCCCCTTGAGCATCTGACTTCTGGGTAATAAAGGAAATTTA
TTTTCATTGCAATAGTGTGTGGGAATTTTTTGTGTCTCTCACTCGGAAGG
ACATATGGGAGGGCAAATCATTTAAAACATCAGAATGAGTATTTGGTTTA
GAGTTTGGCAACATATGCCATATGCTGGCTGCCATGAACAAAGGTGGCTA
TAAAGAGGTCATCAGTATATGAAACAGCCCCCTGCTGTCCATTCCTTATT
CCATAGAAAAGCCTTGACTTGAGGTTAGATTTTTTTTATATTTTGTTTTG
TGTTATTTTTTTCTTTAACATCCCTAAAATTTTCCTTACATGTTTTACTA
GCCAGATTTTTCCTCCTCTCCTGACTACTCCCAGTCATAGCTGTCCCTCT
TCTCTTATGAACTCGACT

In another embodiment, the invention is a transgenic non-human animal whose genome comprises a stable integration of at least one first transgene that includes at least one nucleic acid sequence encoding at least one Fas-ligand protein operably-linked to at least one tetracycline-inducible promoter and at least one second transgene that includes at least one second nucleic acid sequence encoding at least one member selected from the group consisting of a reverse tetracycline responsive transactivator protein and a tetracycline responsive transactivator protein.

In an embodiment, the second transgene includes at least one second nucleic acid sequence encoding a reverse tetracycline responsive transactivator (rtTA) protein. In another embodiment, the second transgene includes at least one second nucleic acid sequence encoding a tetracycline responsive transactivator (tTA) protein.

In an embodiment, the second transgene includes at least one second nucleic acid sequence encoding a reverse tetracycline responsive transactivator protein, and apoptosis is induced in cells of the animal that express both the first and second transgene when tetracycline (tet) or a tetracycline analog is present. Tetracycline analogs are well known and include, for example, naturally occurring (e.g., chlortetracycline, oxytetracycline, demclocycline) and semi-synthetic (e.g., doxycyline (Dox), lymecycline, meclocycline, methacycline, minocycline, rolitetracycline) analogs.

The nucleic acid sequence encoding the reverse tetracycline responsive transactivator protein or the tetracycline responsive transactivator protein in the second transgene can be operably linked to a cell-specific and/or tissue-specific promoter. Exemplary promoters include, for example, a pancreatic β-cell promoter, an amyloid precursor protein gene promoter, a dystrophin gene promoter, a Clara cell secretory protein gene promoter (e.g., a Clara cell secretory protein-C promoter), a surfactant protein-B gene promoter, a surfactant protein-C gene promoter, an insulin gene promoter, an albumin gene promoter, an alpha Calcium/Calmodulin dependent Protein Kinase II (CamKII) gene promoter, a neuron-specific enolase gene promoter, a retinoblastoma gene promoter, a muscle creatine kinase gene promoter, an alpha myosin heavy chain gene promoter, a TEK tyrosine kinase gene promoter, a Tie receptor tyrosine kinase gene promoter, an immunoglobulin (Ig) heavy chain enhancer, a CD34 gene promoter, an SM22alpha gene promoter, and a glial fibrillary acidic gene promoter.

In an embodiment, the second transgene includes the Clara cell secretory protein gene (e.g., Clara cell secretory protein C gene) promoter and the transgenic non-human animal has a phenotype that includes, for example, an alveolar type II cell apoptosis, a nonciliated bronchial epithelial cell apoptosis, a disrupted alveolar development, a decreased vascular density and/or a postnatal lethality (e.g., the animal dies during the postnatal period) consequent to apoptosis.

Standard techniques for assessing apoptosis are well known and include, for example, terminal deoxynucleotidyl transferase-mediated dUTP-FITC nick-end (TUNEL) labeling, ultrastructural analysis (e.g., electron microscopy) and detection of caspase (e.g., caspase-3) cleavage (e.g., by Western blot analysis), as described herein.

“Disrupted alveolar development,” as used herein, refers to alveolar simplification that resembles the pulmonary pathology of human bronchopulmonary dysplasia. Exemplary features of disrupted alveolar development include large simplified airspaces, a paucity of alveolar septation, and secondary crest formation. Disrupted alveolar development can be detected using standard techniques, such as, for example, microscopy, stereological volumetric techniques and morphometric analysis (e.g., computer-assisted morphometric analysis) of mean cord length (MCL) and radial alveolar count (RAC) in lung samples.

“Decreased vascular density,” as used herein, refers to a vessel density that is at least about 25% reduced in the lungs of the double transgenic non-human animal (i.e., the non-human transgenic animal whose genome comprises the first and second transgenes (e.g., an animal having the genotype CCSP-rtTA⁺/(tetOp)₇-FasL⁺)) compared to a control animal (e.g., a single-transgenic littermate, such as an animal having the genotype CCSP-rtTA⁺/(tetOp)₇-FasL⁻). Standard methods of measuring vascular density have been described (Balasubramaniam V, Mervis CF, Maxey A M, Markham N E, Abman SH: Hyperoxia reduces bone marrow, circulating, and lung endothelial progenitor cells in the developing lung: implications for the patho-genesis of bronchopulmonary dysplasia. Am J Physiol 2007, 292:L1073-L1084).

In another embodiment, the second transgene includes a pancreatic β-cell promoter (e.g., a mouse insulin gene promoter, a rat insulin gene promoter); and the transgenic non-human animal has a phenotype that resembles a diabetic condition consequent to apoptosis.

In a further embodiment, the second transgene includes an amyloid precursor protein gene promoter; and the transgenic non-human animal has a phenotype that resembles an Alzheimer's condition consequent to apoptosis.

In an additional embodiment, the second transgene includes a dystrophin gene promoter; and the transgenic non-human animal has a phenotype that resembles a muscular dystrophy condition consequent to apoptosis.

In another embodiment, the second transgene includes an alpha myosin heavy chain gene promoter; and the transgenic non-human animal has a phenotype that resembles a myocardial infarction consequent to apoptosis.

In another embodiment, the second transgene includes a surfactant protein B gene promoter or a surfactant protein C gene promoter; and the transgenic non-human animal has a phenotype that resembles lung injury consequent to apoptosis.

In an additional embodiment, the second transgene includes an albumin gene promoter; and the transgenic non-human animal has a phenotype that resembles liver failure consequent to apoptosis.

The second transgene in the transgenic non-human animals described herein can have at least one member selected from the group consisting of an alphaCaMKII gene promoter, a neuron-specific enolase gene promoter, a glial fibrillary acidic protein gene promoter and a retinoblastoma gene promoter; and the transgenic non-human animal can have a phenotype that resembles a neurodegenerative brain disorder consequent to apoptosis.

In another embodiment, the second transgene includes a muscle creatine kinase gene promoter; and the transgenic non-human animal has a phenotype that resembles a degenerative muscle disorder consequent to apoptosis.

In still another embodiment, the second transgene includes a TEK gene promoter or Tie gene promoter; and the transgenic non-human animal has a phenotype that resembles endothelial injury consequent to apoptosis.

In a further embodiment, the second transgene includes an immunoglobulin heavy chain enhancer; and the transgenic non-human animal has a phenotype that resembles aplastic anemia and/or bone marrow ablation consequent to apoptosis.

In an additional embodiment, the second transgene includes a CD34 gene promoter; and the transgenic non-human animal has a phenotype that resembles bone marrow ablation, aplastic anemia and/or a hematologic condition consequent to apoptosis.

In another embodiment, the second transgene includes an SM22 alpha gene promoter; and the transgenic non-human animal has a phenotype that resembles a condition associated with dissolution of vascular smooth muscle cells consequent to apoptosis.

The transgenic non-human animal can include at least one cell that co-expresses both the first transgene and the second transgene and subsequently undergoes apoptosis.

In an embodiment, the cell of the transgenic non-human animal that expresses a transgene that includes at least one nucleic acid sequence encoding at least one Fas-ligand protein and undergoes apoptosis is an epithelial tissue cell. For example, the epithelial tissue cell can be a lung epithelial cell, such as at least one member selected from the group consisting of a ciliated lung epithelial cell and a non-ciliated lung epithelial cell (e.g., a nonciliated bronchial epithelial cell). In another embodiment, the epithelial tissue cell can be an alveolar lung epithelial cell (e.g., a type II alveolar lung epithelial cell).

In an embodiment, the cell of the transgenic non-human animal that expresses a transgene that includes at least one nucleic acid sequence encoding at least one Fas-ligand protein and undergoes apoptosis is a cell from a tissue other than epithelium (also referred to as a “non-epithelial cell”). For example, in an embodiment, the cell is a connective tissue cell. Exemplary connective tissue cells include, for example, a chondrocyte.

In an additional embodiment, the cell of the transgenic non-human animal that expresses a transgene that includes at least one nucleic acid sequence encoding at least one Fas-ligand protein and undergoes apoptosis is a nervous tissue cell. Exemplary nervous tissue cells include, for example, a central nervous tissue cell (a brain cell or a spinal cord cell) and a peripheral nervous tissue cell.

In another embodiment, the cell of the transgenic non-human animal that expresses a transgene that includes at least one nucleic acid sequence encoding at least one Fas-ligand protein and undergoes apoptosis is a muscle tissue cell (e.g., a smooth muscle cell, a skeletal muscle cell, a cardiac muscle cell).

In yet another embodiment, the cell of the transgenic non-human animal that expresses a transgene that includes at least one nucleic acid sequence encoding at least one Fas-ligand protein and undergoes apoptosis is a retinal cell (e.g., a retinal epithelial cell, a retinal ganglion cell).

In another embodiment, the invention is a recombinant nucleic acid comprising a nucleotide sequence having at least about 75.0% (e.g., about 76.0%, about 93.0%) identity to a murine Fas-ligand cDNA comprising SEQ ID NO:4 operably-linked to a tetracycline-inducible promoter, wherein the tetracycline-inducible promoter includes at least seven copies of a tetracycline operator nucleic acid sequence and a cytomegalovirus minimal promoter nucleic acid sequence. For example, the recombinant nucleic acid can include a nucleic acid sequence encoding a Fas-ligand protein having at least about 75.0%, about 80.0%, about 85.0%, about 90.0%, about 95.0% or about 99.0% identity to SEQ ID NO:4.

The recombinant nucleic acid also can include at least about 7, about 8, about 9, about 10, about 11, about 12, about 13, or about 14 tet operator nucleic acid sequences. In an embodiment, the tetracycline-inducible promoter can include seven copies of the tet operator nucleic acid sequence (e.g., SEQ ID NO:7).

The recombinant nucleic acid can further include a cytomegalovirus minimal promoter nucleic acid sequence. An exemplary cytomegalovirus minimal promoter nucleic acid sequence is SEQ ID NO:8.

In addition, the recombinant nucleic acid can include a polyadenylation nucleic acid sequence (e.g., SEQ ID NO:9).

In an embodiment, the recombinant nucleic acid can include SEQ ID NO: 15, which includes the full-length mouse FasL cDNA and restriction sites used to generate the FasL transgene construct described herein:

(SEQ ID NO: 10)

TCTAGAGAGAAGGAAACCCTTTCCTGGGGCTGGGTGCCATGCAGCAGCCC
ATGAATTACCCATGTCCCCAGATCTTCTGGGTAGACAGCAGTGCCACTTC
ATCTTGGGCTCCTCCAGGGTCAGTTTTTCCCTGTCCATCTTGTGGGCCTA
GAGGGCCGGACCAAAGGAGACCGCCACCTCCACCACCACCTGTGTCACCA
CTACCACCGCCATCACAACCACTCCCACTGCCGCCACTGACCCCTCTAAA
GAAGAAGGACCACAACACAAATCTGTGGCTACCGGTGGTATTTTTCATGG
TTCTGGTGGCTCTGGTTGGAATGGGATTAGGAATGTATCAGCTCTTCCAC
CTGCAGAAGGAACTGGCAGAACTCCGTGAGTTCACCAACCAAAGCCTTAA
AGTATCATCTTTTGAAAAGCAAATAGCCAACCCCAGTACACCCTCTGAAA
AAAAAGAGCCGAGGAGTGTGGCCCATTTAACAGGGAACCCCCACTCAAGG
TCCATCCCTCTGGAATGGGAAGACACATATGGAACCGCTCTGATCTCTGG
AGTGAAGTATAAGAAAGGTGGCCTTGTGATCAACGAAACTGGGTTGTACT
TCGTGTATTCCAAAGTATACTTCCGGGGTCAGTCTTGCAACAACCAGCCC
CTAAACCACAAGGTCTATATGAGGAACTCTAAGTATCCTGAGGATCTGGT
GCTAATGGAGGAGAAGAGGTTGAACTACTGCACTACTGGACAGATATGGG
CCCACAGCAGCTACCTGGGGGCAGTATTCAATCTTACCAGTGCTGACCAT
TTATATGTCAACATATCTCAACTCTCTCTGATCAATTTTGAGGAATCTAA
GACCTTTTTCGGCTTGTATAAGCTTTAAAAGAAAAAGCATTTTAAAATGA
TCTACTATTCTTTATCATGGGCACCAGGAATATTCTAGAGC

In an additional embodiment, the recombinant nucleic acid can include a vector nucleic acid sequence (e.g., a pTRE2 vector (BD Biosciences, Franklin Lakes, N.J.) nucleic acid sequence).

In a further embodiment, the invention is a method for producing a transgenic non-human animal, comprising the step of crossing a first transgenic non-human animal whose genome comprises a stable integration of at least one first transgene that includes at least one nucleic acid sequence encoding at least one Fas-ligand protein operably-linked to a tetracycline-inducible promoter with a second transgenic non-human animal whose genome comprises a stable integration of at least one second transgene that includes at least one second nucleic acid sequence encoding at least one reverse tetracycline responsive transactivator protein.

In an embodiment, at least one cell of the transgenic non-human animal that co-expresses both the first transgene and the second transgene undergoes apoptosis.

The first and second transgenic non-human animals employed in the methods described herein can have a genotype that is hemizygous or homozygous for their respective transgenes.

In an embodiment, both the first transgenic non-human animal and second transgenic non-human animal are mice, such as mice with an FVB/N genetic background.

When the first transgenic non-human animal and second transgenic non-human animal are mice, the second transgenic mouse can be a tet-activator mouse, including mouse lines listed in Table 1.

TABLE 1
General and Tissue-Specific tet-Activator Mouse Lines

| Promoter/ | | Technique | | |
|---|---|---|---|---|
| Transcription | | (tg = transgenic, | | |
| unit | Activator | ki = knock-in) | Tissue Specificity | Reference(s) |
| CMV | tTA rtTA | tg | diverse tissues | Kistner et al., Proc |
| | | | | Natl Acad Sci USA. |
| | | | | 1996 October 1; |
| | | | | 93(20): 10933-10938. |
| hSP-C (surfactant | rtTA | tg | respiratory | Tichelaar et al., J |
| protein-C) | | | epithelium | Biol Chem, Vol. |
| | | | | 275, Issue 16, |
| | | | | 11858-11864, Apr. |
| | | | | 21, 2000 |
| rCcsp (Clara cell | rtTA | tg | respiratory | Tichelaar et al., J |
| secretory protein) | | | epithelium | Biol Chem, Vol. |
| | | | | 275, Issue 16, |
| | | | | 11858-11864, Apr. |
| | | | | 21, 2000 |
| rCC10 (Clara cell | rtTA tTS | tg | lung (Clara cells) | Ray et al., J Clin |
| 10 kDa protein) | | | | Invest. 1997 |
| | | | | November 15; |
| | | | | 100(10): 2501-2511. |
| | | | | Zhu et al., J Biol |
| | | | | Chem. 2001 Jul |
| | | | | 6; 276(27): 25222-9. |
| | | | | Epub 2001 Apr 30. |
| bOpsin | rtTA | tg | photoreceptors | Chang et al., |
| | | | | Investigative |
| | | | | Ophthalmology and |
| | | | | Visual Science. |
| | | | | 2000; 41: 4281-4287 |
| mIRBP | rtTA | tg | photoreceptors | Chang et al., |
| (interphotoreceptor | | | | Investigative |
| retinoid-binding | | | | Ophthalmology and |
| protein) | | | | Visual Science. |
| | | | | 2000; 41: 4281-4287 |
| αCaMKII | tTA rtTA | tg | brain (neurons of | Mayford et al., |
| | | | neocortex, | Science. 1996 Dec |
| | | | hippocampus, | 6; 274(5293): 1678-83. |
| | | | amygdala, basal | Mansuy et al., 1998 |
| | | | ganglia) | |
| NSE (neuron | tTA | tg | striatum, | Chen et al., Neuron. |
| specific enolase) | | | cerebellum, CA1- | 1998 |
| | | | region of | Aug; 21(2): 257-65. |
| | | | hippocampus, | |
| | | | neocortex (deep | |
| | | | layers) | |
| rGH (growth | rtTA | tg | pituitary gland | Roh et al., Mol |
| hormone) | | | (somatotropes) | Endocrinol. 2001 |
| | | | | Apr; 15(4): 600-13. |
| rPRL (prolactin) | tTA | tg | pituitary gland | Roh et al., Mol |
| | | | (lactotropes) | Endocrinol. 2001 |
| | | | | Apr; 15(4): 600-13. |
| PrP (prion protein) | tTA | tg | brain (neocortex, | Tremblay et al., |
| | | | entorhinal cortex, | Proc Natl Acad Sci |
| | | | hippocampus, | USA. 1998 |
| | | | subst. nigra, | October 13; 95(21): |
| | | | thalamus, | 12580-12585 |
| | | | cerebellum) | |
| rNestin | rtTA | tg | neuroepithelium | Mitsuhashi et al., |
| (tog. with IRES-β- | | | (tel-, mes-, | Proc Natl Acad Sci |
| geo) | | | rhombencephalon, | USA. 2001 May |
| | | | spinal cord, | 22; 98(11): 6435-6440. |
| | | | retina, trigeminal | |
| | | | ganglion) | |
| hRB | rtTA | tg | retinal ganglion | Utomo et al., Nat |
| (retinoblastoma) | | | layer neurons, | Biotechnol. 1999 |
| (tog. with tetO- | | | cerebellar | Nov; 17(11): 1091-6. |
| CRE) | | | Purkinje cells, | |
| | | | thalamus, | |
| | | | myocytes of thigh | |
| | | | muscles | |
| hPmp22 | tTA | tg (YAC) | Schwann cells | Perea et al., Human |
| (peripheral myelin | | | | Molecular |
| protein 22) | | | | Genetics, 2001, |
| | | | | Vol. 10, No. 10 |
| | | | | 1007-1018 |
| mip2 (proinsulin | rtTA | tg | pancreatic β-cells | Lottmann et al., J |
| gene II promoter) | | | | Mol Med. 2001 |
| (tog. with tetO- | | | | Jun; 79(5-6): 321-8. |
| PDX-1as) | | | | |
| rip (rat insuline II | rtTA | tg | pancreatic β-cells | Thomas et al., J |
| promoter) | | | | Clin Invest. 2001 |
| | | | | Jul; 108(2): 319-29. |
| mAlb (albumin) | tTA | tg | liver | Manickan et al., J |
| | | | | Biol Chem. 2001 |
| | | | | Apr |
| | | | | 27; 276(17): 13989-94. |
| mMup (major | tTA | tg | liver | Manickan et al., J |
| urinary protein) | | | | Biol Chem. 2001 |
| | | | | Apr |
| | | | | 27; 276(17): 13989-94. |
| rLAP (liver | tTA | tg | liver | Kistner et al., Proc |
| enriched activator | | | | Natl Acad Sci USA. |
| protein) | | | | 1996 October 1; |
| | | | | 93(20): 10933-10938. |
| bk5 (keratin 5) | tTA rtTA | tg | skin (epidermal | Diamond et al., J |
| | | | basement layer | Invest Dermatol. |
| | | | and hair follicles) | 2000 |
| | | | | Nov; 115(5): 788-94. |
| bK6 (keratin 6) | tTA | tg | skin | Guo et al., Mol |
| | | | | Carcinog. 1999 |
| | | | | Sep; 26(1): 32-6. |
| hK14 (keratin 14) | rtTA | tg | cornified and non- | Xie et al., |
| | | | cornified | Oncogene. 1999 |
| | | | squamous | Jun |
| | | | epithelia (skin, | 17; 18(24): 3593-607. |
| | | | esophagus, | |
| | | | tongue, cornea) | |
| hK18 (keratin 18) | rtTA | tg | trachea, bronchi, | Ye et al., Mol Ther. |
| | | | lungs, submucosal | 2001 May; 3(5 Pt |
| | | | glands | 1): 723-33. |
| mTyr (tyrosinase) | rtTA | tg | melanocytes | Chin et al., Nature. |
| | | | | 1999 Jul |
| | | | | 29; 400(6743): 468-72. |
| mEdnrb | tTA rtTA | ki | melanocytes, | Shin et at., Nature. |
| (endothelin | | | enteric neurons | 1999 Dec |
| receptor B) | | | | 2; 402(6761): 496-501. |
| Fabpl^{4xat-132} (fatty | rtTA | tg | small intestine, | Saam and Gordon, |
| acid binding | | | cecum, colon, | J Biol Chem. 1999 |
| protein) | | | bladder | Dec |
| | | | | 31; 274(53): 38071-82. |
| rWap (whey acidic | rtTA | tg | mammary | Utomo et al., Nat |
| protein) | | | epithelial cells, | Biotechnol. 1999 |
| (tog. with tetO- | | | (kidney | Nov; 17(11): 1091-6. |
| CRE) | | | glomeruli) | |
| MMTV-LTR | tTA | tg | mammary gland, | Hennighausen et |
| | | | salivary gland, | al., J Cell Biochem. |
| | | | seminal vesicle, | 1995 |
| | | | Leydig cells, bone | Dec; 59(4): 463-72. |
| | | | marrow (brain, | |
| | | | kidney, liver, | |
| | | | spleen) | |
| MMTV-LTR | rtTA | tg | mammary gland, | D'Cruz et al., Nat |
| | | | salivary gland, | Med. 2001 |
| | | | seminal vesicle | Feb; 7(2): 235-9. |
| | | | | Gunther et al., |
| | | | | FASEB J. 2002 |
| | | | | Mar; 16(3): 283-92. |
| β-lactoglobulin | rtTA | tg | mammary gland | Soulier et al., Eur J |
| (tog. with tetO- | | | | Biochem. 1999 |
| αlactalbumin) | | | | Mar; 260(2): 533-9. |
| hMCK (muscle | tTA | tg | striated muscle | Ghersa et al., Gene |
| creatine kinase) | | | | Ther. 1998 |
| | | | | Sep; 5(9): 1213-20. |
| MCK (muscle | tTA | tg | striated muscle | Ahmad et al., Hum |
| creatine kinase | | | | Mol Genet. 2000 |
| | | | | Oct 12; 9(17): 2507-15. |
| rαMHC (alpha | tTA rtTA | tg | cardiac myocytes | Yu et al., Circ Res. |
| myosin heavy | | | | 1996 |
| chain) | | | | Oct; 79(4): 691-7. |
| | | | | Passman and |
| | | | | Fishman, J Clin |
| | | | | Invest. 1994 |
| | | | | Dec; 94(6): 2421-5. |
| | | | | Valencik and |
| | | | | McDonald, |
| | | | | Transgenic Res. |
| | | | | 2001 |
| | | | | Jun; 10(3): 269-75. |
| SM22α | tTA | tg | vascular smooth | Ju et al., Proc Natl |
| | | | muscle cells | Acad Sci USA. |
| | | | | 2001 Jun |
| | | | | 19; 98(13): 7469-74. |
| mTek | tTA | tg | endothel | Sarao et al., |
| | | | (embryonic) | Transgenic Res. |
| | | | | 1998 Nov; 7(6): 421-7. |
| mTie | tTA | tg | endothel (adult) | Sarao et al., |
| | | | | Transgenic Res. |
| | | | | 1998 Nov; 7(6): 421-7. |
| Ig heavy chain- | tTA | tg | hematopoietic | Felsher et al., Mol |
| enhancer/SRα- | | | cells | Cell. 1999 |
| promoter | | | | Aug; 4(2): 199-207. |
| Ig heavy chain- | tTA | tg | developing B- and | Hess et al., Mol |
| enhancer/minimal | | | T-cells in spleen | Cell Biol. 2001 |
| promoter | | | and thymus | Mar; 21(5): 1531-9. |
| | | | (lymphopoiesis); | |
| | | | skeletal muscle | |
| hCD2 | rtTA | tg | T-cells | Legname et al., |
| | | | | Immunity. 2000 |
| | | | | May; 12(5): 537-46. |
| Lck | tTA | tg | T-cells | Leenders et al., Eur |
| | | | | J Immunol. 2000 |
| | | | | Oct; 30(10): 2980-90. |
| MHCIIEα^{κ} | tTA | tg | thymic epithelial | Witherden et al., J |
| | | | cells | Exp Med. 2000 Jan |
| | | | | 17; 191(2): 355-64. |
| murine SM22 | rtTA | tg | smooth muscle | West et al. Circ |
| promoter | | | | Res. 2004 Apr |
| | | | | 30; 94(8): 1109-14. |
| | | | | Epub |
| Foxg1 genomic | tTA | ki | brain | Hanashima et al. |
| locus | | | (progenitors) | Science. 2004 Jan |
| | | | | 2; 303(5654): 56-9. |
| humanCD34 YAC | tTA | tg | haematopoietic | Huettner et al., |
| | | | stem cells, CMPs | Blood. 2003 Nov |
| | | | and MEPs, | 1; 102(9): 3363-70. |
| | | | megakaryocytes | |
| surfactant protein | rtTA | tg | lung | Mucenski et al. J |
| C promoter | | | | Biol Chem. 2003 |
| | | | | Oct |
| | | | | 10; 278(41): 40231-8. |
| Clara cell | rtTA | tg | lung | Mucenski et al. J |
| secretory protein | | | | Biol Chem. 2003 |
| promoter | | | | Oct |
| | | | | 10; 278(41): 40231-8. |
| tyrosinase | rtTA | tg | retina | Lavado et al. Front |
| promoter and | | | | Biosci. 2006 Jan |
| Locus Control | | | | 1; 11: 743-52 |
| Region (LCR) | | | | |
| liver activator | rtTA | tg | kidney and liver | Gallagher et al. J |
| protein (LAP) | | | | Am Soc Nephrol. |
| promoter | | | | 2003 |
| | | | | Aug; 14(8): 2042-51. |
| human podocin | rtTA | tg | podocytes in the | Shigehara et al., J |
| (NPHS2) gene | | | kidney | Am Soc Nephrol. |
| promoter | | | | 2003 |
| | | | | Aug; 14(8): 1998-2003. |
| GABA(A) | rtTA | tg | cerebellar granule | Yamamoto et al., J |
| receptor alpha6 | | | cells of the brain | Neurosci. 2003 Jul |
| gene promoter | | | | 30; 23(17): 6759-67. |
| small-conductance | tTA | ki | mesenteric | Taylor et al., Circ |
| Ca2⁺ activated K⁺ | | | arteries of the | Res. 2003 Jul |
| channel | | | endothelium | 25; 93(2): 124-31. |
| Tet-promoter | tTA | tg | various tissues | Hwang et al., Arch |
| | | | | Biochem Biophys. |
| | | | | 2003 Jul |
| | | | | 15; 415(2): 137-45 |
| rat Clara cell | rtTA | tg | lung | Yan et al., |
| secretory protein | | | | Endocrinology. |
| 2.3-kb promoter | | | | 2003 |
| | | | | Jul; 144(7): 3004-11. |
| human b-actin | tTA | tg | various tissues | Lamposa et al., |
| promoter | | | | FASEB J. 2003 |
| | | | | Jul; 17(10): 1343-5 |
| mouse muscle | rtTA | tg | skeletal muscle | Grill et al., |
| creatine kinase | | | | Transgenic Res. |
| (MCK) promoter | | | | 2003 Feb; 12(1): 33-43 |
| major immediate- | tTA | tg | multiple tissues | Haribhai et al., J |
| early human CMV | | | | Immunol. 2003 |
| promoter and | | | | Mar |
| enhancer | | | | 15; 170(6): 3007-14. |
| hCMV | rtTA | tg | multiple tissues | Manfra et al., J |
| enhancer/chicken | | | but not in | Immunol. 2003 |
| β-actin promoter | | | haemopoietic | Mar |
| | | | organs | 15; 170(6): 2843-52 |
| mouse alpha- | tTA/rtTA | tg | heart | Sanbe et al., Circ |
| myosin heavy | | | | Res. 2003 Apr |
| chain promoter | | | | 4; 92(6): 609-16 |
| MMTV-LTR | rtTA | tg | mammary gland | Gunther et al., |
| | | | | Genes Dev. 2003 |
| | | | | Feb 15; 17(4): 488-501 |
| SM22α promoter | tTA | tg | vascular SMCs of | You et al., Circ |
| | | | aorta, carotid, | Res. 2003 Feb |
| | | | mesentery, liver, | 21; 92(3): 314-21 |
| | | | lung, kidney, and | |
| | | | spleen | |
| murine rhodopsin | tTA | tg | photoreceptor | Angeletti et al., |
| promoter | | | cells of the outer | Invest Ophthalmol |
| | | | nuclear layer of | Vis Sci. 2003 |
| | | | the retina | Feb; 44(2): 755-60 |
| rat insulin | tTA | tg | beta-cells of the | Christen et al., |
| promoter | | | pancreas | Transgenic Res. |
| | | | | 2002 |
| | | | | Dec; 11(6): 587-95 |
| small-conductance | tTA | tg | magnocellular | Bond et al., |
| Ca²⁺-activated | | | neurons of the | Science. 2000 Sep |
| potassium channel | | | supraoptic | 15; 289(5486): 1942-6 |
| (SK channel) | | | nucleus and in | |
| | | | many smooth | |
| | | | muscles, | |
| | | | including the | |
| | | | uterus | |
| Clara cell-specific | rtTA | tg | lung, airways | Mehrad et al., Am J |
| CC10 promoter | | | | Respir Crit Care |
| | | | | Med. 2002 Nov |
| | | | | 1; 166(9): 1263-8 |
| human SP-C | rtTa | tg | peripheral | Clark et al., Am J |
| promoter | | | respiratory | Physiol Lung Cell |
| | | | epithelial cells in | Mol Physiol. 2001 |
| | | | the lungs of fetal | Apr; 280(4): L705-15 |
| | | | [postconception | Pearl et al., |
| | | | (pc) day 15] and | Transgenic Res. |
| | | | adult mice | 2002 Feb; 11(1): 21-9 |
| rat CCSP promoter | rtTA | tg | tracheobronchial | Clark et al., Am J |
| | | | and type II cells | Physiol Lung Cell |
| | | | | Mol Physiol. 2001 |
| | | | | Apr; 280(4): L705-15 |
| | | | | Pearl et al., |
| | | | | Transgenic Res. |
| | | | | 2002 Feb; 11(1): 21-9 |
| tek/Tie2 | rtTA | tg | endothelial cells | Teng et al., Physiol |
| promoter/intron | | | | Genomics. 2002 |
| enhancer | | | | Oct 29; 11(2): 99-107 |
| Pdx1 gene | tTA | ki | pancreas | Holland et al., Proc |
| | | | | Natl Acad Sci USA. |
| | | | | 2002 Sep |
| | | | | 17; 99(19): 12236-41 |
| immunoglobulin | tTA | tg | haematopoietic | Jain et al., Science. |
| heavy chain | | | cells and | 2002 Jul |
| enhancer and the | | | immature | 5; 297(5578): 102-4 |
| SRa promoter | | | osteoblasts | Felscher et al., Mol |
| (EmSR) | | | | Cell. 1999 |
| | | | | Aug; 4(2): 199-207 |
| neuron-specific | tTA | tg | brain | Sakai et al., Mol |
| enolase (NSE) | | | | Pharmacol. 2002 |
| promoter | | | | Jun; 61(6): 1453-64 |
| CMV | rtTA | tg | high levels of | Wiekowski et al., J |
| enhancer/chicken^{β}- | | | expression in | Immunol. 2001 Dec |
| actin promoter | | | heart; moderate | 15; 167(12): 7102-10 |
| | | | expression levels | |
| | | | in skin, kidney, | |
| | | | thymus, and lung; | |
| | | | and low | |
| | | | expression levels | |
| | | | in spleen and liver | |
| human K14 | tTA | tg | mammary gland | Dunbar et al., J |
| promoter | | | | Endocrinol. 2001 |
| | | | | Dec; 171(3): 403-16 |
| mouse albumin | tTa | tg | skeletal muscle, | Raben et al., Hum |
| promoter/enhancer | | | heart, liver, lung | Mol Genet. 2001 |
| | | | and spleen | Sep |
| | | | | 15; 10(19): 2039-47 |
| Lck proximal | tTA | tg | lymphoid organs | Labrecque et al., |
| promoter | | | (with the | Immunity. 2001 |
| | | | exception of a | Jul; 15(1): 71-82 |
| | | | weak signal in the | |
| | | | ovaries) | |
| mouse proinsulin | rtTA | tg | ^{β}-cells of | Lottmann et al., J |
| gene II promoter | | | pancreatic islets. | Mol Med. 2001 |
| | | | | Jun; 79(5-6): 321-8 |
| hCMV | tTA/rtTA | tg in ES cells | multiple tissues | Fedorov et al., |
| | | | | Transgenic Res. |
| | | | | 2001 |
| | | | | Jun; 10(3): 247-58 |
| prion protein (PrP) | tTA | tg | neuronal and glial | Gotz et al., Eur J |
| promoter | | | cells | Neurosci. 2001 |
| | | | | Jun; 13(11): 2131-40 |
| surfactant protein | rtTA | tg | lung epithelial | Perl et al. Proc Natl |
| C promoter | | | cells | Acad Sci USA. |
| | | | | 2002 Aug |
| | | | | 6; 99(16): 10482-7 |
| | | | | Nguyet et al. Dev |
| | | | | Biol. 2005 Jun |
| | | | | 1; 282(1): 111-25 |
| SM22a promoter | rtTA | tg | arterial smooth | Bernal-Mizrachi et |
| | | | muscle and brown | al. Nature. 2005 |
| | | | fat tissue | May |
| | | | | 26; 435(7041): 502-6 |
| keratin 12 gene | rtTA | ki | corneal | Chikama et al. |
| locus | | | epithelium | Invest Ophthalmol |
| | | | | Vis Sci. 2005 |
| | | | | Jun; 46(6): 1966-72 |
| liver-specific | rtTA | tg | liver | Xu et al. World J |
| human apoE | | | | Gastroenterol. 2005 |
| promoter | | | | May |
| | | | | 21; 11(19): 2885-91 |
| platelet factor 4 | rtTA | tg | megakaryocytes | Nguyen et al. |
| promoter | | | and platelets | Blood. 2005 Sep |
| | | | | 1; 106(5): 1559-64 |
| Vav promoter | tTA | tg | haematopoietic, | Wiesner et al. |
| | | | mast cells | Blood. 2005 Aug |
| | | | | 1; 106(3): 1054-62 |
| nestin promoter (5- | rtTA-M2- | tg | subventricular | Yu et al. Genesis. |
| kb nestin promoter | GFP | | zone and the | 2005 |
| and the 700-bp | | | dentate gyrus | Apr; 41(4): 147-53 |
| second intron of | | | | |
| the nestin gene) | | | | |
| murine keratocan | rtTA | tg | cornea stromal | Hayash et al. Mol |
| promoter | | | keratocytes | Vis. 2005 Mar |
| | | | | 16; 11: 201-7 |
| bovine keratin 5 | NLS- | tg | basal layer of | Vitale-Cross et al. |
| (K5) promoter | rtTA | | stratified | Cancer Res. 2004 |
| | | | epithelium | Dec |
| | | | including the | 15; 64(24): 8804-7 |
| | | | epithelial stem | |
| | | | cells | |
| mouse major | tTA | tg | liver | Manickan et al. J |
| urinary protein | | | | Biol Chem. 2001 |
| promoter | | | | Apr |
| | | | | 27; 276(17): 13989-94 |
| | | | | Lindeberg et al. J |
| | | | | Neurosci Res. 2002 |
| | | | | Apr 15; 68(2): 248-53 |
| Glial fibrillary | tTA | tg | astrocytes | Wang et al. Mol |
| acidic protein | | | | Cell Neurosci. 2004 |
| (GFAP) | | | | Dec; 27(4): 489-96 |
| SOX 10 gene | rtTA | ki | emerging neural | Ludwig et al. |
| locus | | | crest, several of | Genesis. 2004 |
| | | | its derivatives and | Nov; 40(3): 171-5 |
| | | | oligodendrocytes | |
| human VMD2 | rtTA | tg | retinal pigmented | Oshima et al. J Cell |
| promoter | | | epithelial cells | Physiol. 2004 |
| | | | | Dec; 201(3): 393-400 |
| human Involucrin | tTA/rtTA | tg | epidermis | Jaubert et al. J |
| promoter | | | | Invest Dermatol. |
| | | | | 2004 |
| | | | | Aug; 123(2): 313-8 |
| nestin promoter | tTA | tg | subventricular | Beech et al. J Comp |
| and enhancer | | | zone and rostral | Neurol. 2004 Jul |
| | | | migratory stream, | 12; 475(1): 128-41 |
| | | | periglomerular | |
| | | | neurons in the | |
| | | | brain | |
| CMV | rtTA | tg | most tissues | Wiekowski et al. J |
| enhancer/chicken | | | | Immunol. 2001 Dec |
| beta-globin | | | | 15; 167(12): 7102-10 |
| promoter | | | | |
| Stem cell | tTA-2S | ki | lineage negative, | Bockamp et al. |
| leukaemia gene | | | c-kit espression | Blood. 2006 Sep |
| locus | | | blood stem cells, | 1; 108(5): 1533-41. |
| | | | erythrocytes, | |
| | | | megakaryocytes, | |
| | | | granulocytes | |
| Albumin/MUP | tTA | tg | liver | Manickan et al. J |
| (mouse urinary | | | | Biol Chem. 2001 |
| protein | | | | Apr |
| promoter) | | | | 27; 276(17): 13989-94. |
| Growth hormone | tTA/rtTA | | pituitary glandl, | Roth et al. Mol |
| (GH) | | | lactotrope- | Endocrinol. 2001 |
| PRL-promoter | | | specific | Apr; 15(4): 600-13. |
| | | | expression | |

In an additional embodiment, the invention is a method of screening for a compound that inhibits Fas-ligand mediated apoptosis, comprising the step of assessing Fas-ligand mediated apoptosis in a transgenic non-human animal whose genome comprises a stable integration of at least one first transgene that includes at least one nucleic acid sequence encoding at least one Fas-ligand protein operably-linked to at least one tetracycline-inducible promoter and at least one second transgene that includes at least one second nucleic acid sequence encoding at least one reverse tetracycline responsive transactivator protein, wherein the Fas-ligand mediated apoptosis is in response to administration of the compound in combination with at least one member selected from the group consisting of a tetracycline and a tetracycline analog to the transgenic non-human animal.

Exemplary compounds for use in the method include, for example, small molecules (e.g., small organic molecules, small inorganic molecules), peptides, peptidomimetics, polypeptides (e.g., fusion proteins, antibodies, antibody fragments), nucleic acids (e.g., siRNA, aptamers). In different embodiments, the compound can be FasL siRNA, caspase (e.g., caspase-3, caspase-6) siRNA, caspase small molecule inhibitors, or a FasL fusion protein.

In another embodiment, the invention is a method of screening for a compound that promotes Fas-ligand mediated apoptosis, comprising the step of assessing Fas-ligand mediated apoptosis in a transgenic non-human animal whose genome comprises a stable integration of at least one first transgene that includes at least one nucleic acid sequence encoding at least one Fas-ligand protein operably-linked to at least one tetracycline-inducible promoter and at least one second transgene that includes at least one second nucleic acid sequence encoding at least one reverse tetracycline responsive transactivator protein, wherein the Fas-ligand mediated apoptosis is in response to administration of the compound in combination with at least one member selected from the group consisting of a tetracycline and a tetracycline analog to the transgenic non-human animal.

In a further embodiment, the invention is a method of identifying a cell that is capable of differentiating into a target cell, comprising the steps of inducing apoptosis of a population of target cells in a transgenic non-human animal whose genome comprises a stable integration of at least one first transgene that includes at least one nucleic acid sequence encoding at least one Fas-ligand protein operably-linked to at least one tetracycline-inducible promoter and at least one second transgene that includes a second nucleic acid sequence encoding at least one member selected from the group consisting of a reverse tetracycline responsive transactivator protein and a tetracycline responsive transactivator protein, and wherein the first transgene and the second transgene are co-expressed in the target cells; introducing at least one cell into the transgenic non-human animal, wherein the cell is selected form the group consisting of a stem cell, a progenitor cell and a bone marrow-derived cell; and detecting differentiation of the cell into a phenotype characteristic of the target cell.

Exemplary target cells include pancreatic β-cells, cardiac muscle cells, skeletal muscle cells, neurons, glial cells and lung epithelial cells.

In an embodiment, the cell introduced into the transgenic non-human animal can be a stem cell. The stem cell can be a bone marrow-derived stem cell, such as a hematopoietic stem cell (e.g., a hemocytoblast) or a nonhematopoietic stem cell (e.g., a bone marrow-derived mesenchymal cell, a bone marrow-derived stromal cell, a bone marrow-derived macrophage cell, a bone marrow-derived dendritic cell).

In another embodiment, the cell introduced into the transgenic non-human animal can be a progenitor cell. Exemplary progenitor cells include a satellite cell, an intermediate progenitor cell, a neural progenitor cell, a periosteal cell, a pancreatic progenitor cell, and endothelial progenitor cell.

Differentiation of the cell into a phenotype characteristic of the target cell can be determined by standard techniques (e.g., microscopy, histological evaluation, immunocytochemical analysis).

In an embodiment, differentiation of the cell into a phenotype characteristic of the target cell can be determined, for example, by detecting repopulation of the target cells in the animal.

### EXEMPLIFICATION

Premature infants are at risk for bronchopulmonary dysplasia, a complex condition characterized by impaired alveolar development and increased alveolar epithelial apoptosis. The functional involvement of pulmonary apoptosis in bronchopulmonary dysplasia-associated alveolar disruption remains undetermined. The aims of this study were to generate conditional lung-specific Fas-ligand (FasL) transgenic mice and to determine the effects of FasL-induced respiratory epithelial apoptosis on alveolar remodeling in postcanalicular lungs. Transgenic (TetOp)7-FasL responder mice, generated by pronuclear microinjection, were bred with Clara cell secretory protein (CCSP)-rtTA activator mice. Doxycycline (Dox) was administered from embryonal day 14 to postnatal day 7, and lungs were studied between embryonal day 19 and postnatal day 21. Dox administration induced marked respiratory epithelium-specific FasL mRNA and protein up-regulation in double-transgenic CCSP-rtTA⁺/(TetOp)7-FasL⁺ mice compared with single-transgenic CCSP-rtTA⁺ littermates. The Dox-induced FasL up-regulation was associated with dramatically increased apoptosis of alveolar type II cells and Clara cells, disrupted alveolar development, decreased vascular density, and increased postnatal lethality. The data described herein demonstrate that FasL-induced alveolar epithelial apoptosis during postcanalicular lung remodeling is sufficient to disrupt alveolar development after birth. The availability of inducible lung-specific FasL transgenic mice will facilitate studies of the role of apoptosis in normal and disrupted alveologenesis and may lead to novel therapeutic approaches for perinatal and adult pulmonary diseases characterized by dysregulated apoptosis.

Preterm infants who require assisted ventilation and supplemental oxygen are at risk for bronchopulmonary dysplasia (BPD), a chronic lung disease of newborn infants associated with significant mortality and long-term morbidity.^{1,2} The pathological hallmark of BPD in the postsurfactant era is an impairment of alveolar development, resulting in large and simplified airspaces that show little evidence of vascularized ridges (secondary crests) or alveolar septa.^{3,4} In addition, the lungs of infants with BPD show structurally abnormal microvasculature and variable degrees of interstitial fibrosis.^{5,6} The BPD currently observed in extremely premature infants has been termed “new” BPD to differentiate this condition from the pathologically and epidemiologically distinct historical BPD, originally described in the late 1960s by Northway and colleagues.⁷ The latter occurred in less premature infants and was characterized by more severe patterns of acute lung and airway injury.

Many risk factors have been implicated in the pathogenesis of BPD. Among these, prematurity, oxygen toxicity, and barotrauma are considered central to a final common outcome.^{1,8} There are variable contributions of infection/inflammation, glucocorticoid exposure, chorioamnionitis, and genetic polymorphisms.^{1,9} The precise mechanisms whereby these predisposing conditions result in disrupted alveolar development remain primarily unknown.

Our research efforts in recent years have focused on the role of alveolar epithelial apoptosis in postcanalicular alveolar development. It has been demonstrated that moderate and precisely timed alveolar epithelial type II cell apoptosis is an integral component of physiological postcanalicular lung remodeling in mice, rats, and rabbits. Although the exact biological role of apoptosis in alveologenesis remains uncertain, its choreographed occurrence across mammalian species strongly suggests apoptotic elimination of surplus type II cells during perinatal alveolar remodeling is a naturally occurring and developmentally relevant event.

Although moderate and well timed apoptosis appears to represent a physiological phenomenon during postcanalicular lung development, exaggerated and/or premature alveolar epithelial apoptosis may play a critical role in the pathogenesis of BPD-associated alveolar disruption. Several recent reports described increased levels of alveolar epithelial apoptosis in the lungs of ventilated preterm infants with respiratory distress syndrome or early BPD.¹⁵⁻¹⁷ The temporal patterns of apoptosis and alveolar disruption in ventilated preterm lungs are suggestive of a causative relationship; however, functional involvement of alveolar epithelial apoptosis in disrupted alveologenesis has not been demonstrated thus far.

As described herein, a gain-of-function approach was used to determine the functional role of alveolar epithelial apoptosis in alveolar remodeling. Enhanced respiratory epithelial apoptosis was achieved by means of a transgenic Fas-ligand (FasL) overexpression system. The Fas/FasL receptor-mediated death-signaling pathway is one of the better characterized apoptotic signaling systems.¹⁸⁻²⁰ Stimulation of the Fas receptor (CD95/APO1), a member of the tumor necrosis factor receptor superfamily, by its natural ligand FasL or by Fas-activating antibody ligands results in its trimerization and the recruitment of two key signaling proteins, the adapter protein Fas-associated death domain and the initiator cysteine protease caspase-8. Subsequent activation of the effector caspases through mitochondria-dependent or -independent pathways results in activation of caspase-3, the key effector caspase. Activated caspase-3 cleaves a variety of substrates, including DNA repair enzymes, cellular and nuclear structural proteins, endonucleases, and many other cellular constituents, culminating in effective cell death.¹⁸⁻²¹

Selection of the Fas/FasL system as inducer of alveolar epithelial apoptosis for this study was a logical choice. First, perinatal murine respiratory epithelial cells were previously demonstrated to be exquisitely sensitive to Fas-mediated apoptosis in vitro and in vivo.^{10,22} Furthermore, the Fas/FasL signaling pathway lends itself better to experimental manipulation than other, in particular intrinsic (mitochondrial-dependent) pathways. Finally, the Fas/FasL system has been implicated as critical regulator of alveolar type II cell apoptosis in physiological alveolar remodeling^{10,11} and in various clinical and experimental models of adult lung injury.²³⁻³³ It is therefore conceivable that Fas/FasL signaling may play an important role in BPD-associated apoptosis as well.

The in vivo effect of Fas-activation in perinatal lungs was previously tested by systemic administration of a Fas-activating antibody to newborn mice.¹⁰ This approach allowed us to demonstrate that perinatal alveolar epithelial cells are susceptible to Fas-mediated apoptosis.¹⁰ However, systemic Fas activation resulted in rapid death from liver failure before the effects of exaggerated alveolar apoptosis on alveolar remodeling could be ascertained. To circumvent the deleterious effects of prolonged and systemic FasL exposure, a tetracycline-inducible (tet-on) lung epithelial-specific FasL-overexpressing mouse was generated, adapted from the Tet system of Gossen and colleagues,³⁴ to target apoptosis to respiratory epithelial cells during perinatal lung development.

The results described herein demonstrate that increased apoptosis of respiratory epithelial cells during postcanalicular alveolar remodeling is sufficient to disrupt alveolar development and results in BPD-like alveolar simplification. These findings support our hypothesis that excessive or premature postcanalicular alveolar epithelial apoptosis is a pivotal event in the pathogenesis of BPD. Elucidation of the role and regulation of postcanalicular alveolar epithelial apoptosis may result in important insights into the regulation of alveologenesis and the pathogenesis of BPD. This, in turn, may open new therapeutic opportunities for the prevention and treatment of this disease, as well as other pulmonary conditions associated with dysregulated alveolar epithelial apoptosis, such as acute lung injury, emphysema, and neoplasia.

Materials and Methods

Generation of a Tetracycline-Dependent Respiratory Epithelium-Specific FasL

### Transgenic Mouse

The tetracycline-inducible system in vivo consists of two independent transgenic mouse lines, an activator line and a responder line. The activator line expresses the reverse tetracycline responsive transactivator (rtTA) in a tissue- or cell-specific manner, whereas the responder line carries a transgene of interest under control of the tet-operator (TetOp). A tet-on tetracycline dependent overexpression system was selected to achieve conditional respiratory epithelium-specific FasL transgene expression. In the tet-on system, transgene expression is induced by binding of the tetracycline analogue, doxycycline (Dox) to rtTA, which in turn activates the (tetOp)7-CMV target promoter, activating transcription of the gene of interest.^{34,35} Tetracycline-dependent transgene expression was targeted to respiratory epithelial cells by using transgenic CCSP-rtTA activator mice in which the rtTA is placed under control of Clara cell secretory protein (CCSP, CC-10) promoter elements.^{36,37} The specific CCSP-rtTA transactivator mice used for these studies have been shown previously to be robust activators that effectively drive transgene expression not only in nonciliated bronchial epithelial (Clara) cells, but also in alveolar type II cells.³⁶⁻⁴² The 2.3-kb rat CCSP promoter element used in these activator mice is thus expressed differently from the native murine CCSP gene, which is limited to Clara cells.⁴³ Consistent with the endogenous expression pattern of CCSP, which is active from embryonal day 12.5 (E12.5) onward, CCSP-rtTA activator mice have been shown to be particularly suitable for studies of gene function in late gestation and postnatal lungs.³⁷ CCSP-rtTA activator mice, generated in a FVB/N background, were a generous gift from Dr. J. Whitsett^{37,38} (Cincinnati Children's Hospital Medical Center, Cincinnati, Ohio).

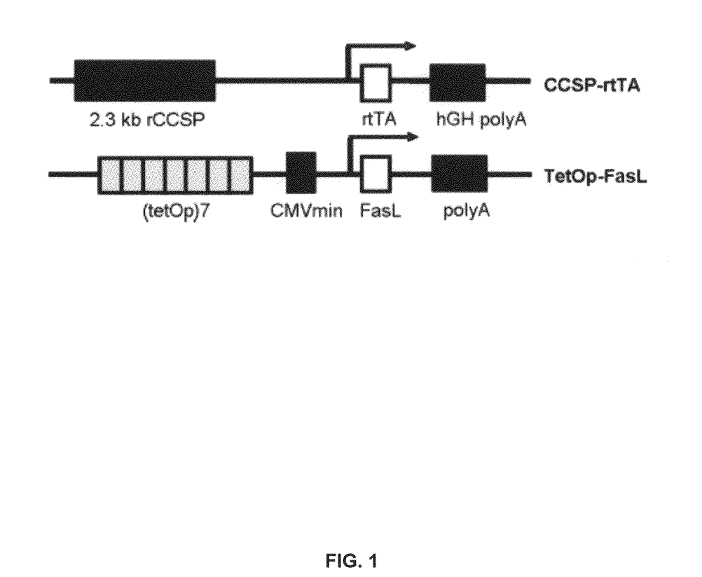

To generate (tetOp)7-FasL responder mice, the 943-bp murine FasL cDNA containing the entire coding region of the protein, a kind gift from Dr. S. Nagata⁴⁴ (Osaka University Medical School, Osaka, Japan), was subcloned between a CMV minimal promoter and bovine growth hormone intronic and polyadenylation sequences in the pTRE2 vector (BD Biosciences, Franklin Lakes, N.J.) (FIG. 1). Restriction enzyme digestion and direct sequencing confirmed the orientation of the insert. The transgene was microinjected into fertilized FVB/N oocytes at the Cincinnati Children's Hospital Transgenic Core Facility. Founders were identified by transgene-specific polymerase chain reaction (PCR) using upstream primer 5′-CGCCTGGAGACGCCATC-3′ (pTRE2) (SEQ ID NO: 11) or 5′-GTGCCATGCAGCAGCCCATGA-3′ (FasL) (SEQ ID NO:12) and downstream primer 5′-CCATTCTAAACAACACCCTG-3′ (pTRE2) (SEQ ID NO:13). Five (tetOp)₇-FasL transgenic mouse lines were expanded. Genotype, transgene copy number, and number of integration sites were determined by Southern blot hybridization initially, and monitored by real-time PCR subsequently (FasL primers, PPM02926A; SuperArray Bioscience Corp., Frederick, Md.).

Animal Husbandry and Tissue Processing

Transgenic (tetOp)₇-FasL progeny derived from founders A through E were crossed with CCSP-rtTA mice to yield a mixed offspring of double-transgenic (CCSP-rtTA⁺/(tetOp)₇-FasL⁺) and single-transgenic (CCSP-rtTA⁺/(tetOp)₇-FasL⁻) littermates. For the sake of brevity, double-transgenic mice will be denoted in the text as CCSP⁺/FasL⁺ mice, whereas single-transgenic mice will be denoted as CCSP⁺/FasL⁻ mice.

Dox (1.0 mg/ml) was added to the drinking water of pregnant and nursing dams between E14 and postnatal day 7 (P7). The progeny (CCSP⁺/FasL⁺ and CCSP⁺/FasL⁻) were sacrificed at E19, P7 (early alveolarization stage⁴⁵), or P21 (late alveolarization stage) by pentobarbital overdose. The cages were inspected twice daily to record interval postnatal death. Body and wet lung weights were recorded. Pups were genotyped by PCR analysis of tail genomic DNA using the primers described above. For molecular analysis, lungs were snap-frozen in liquid nitrogen and stored at −80° C. For morphological studies, fetal lungs were immersion-fixed in freshly prepared 4% paraformaldehyde in phosphate-buffered saline, pH 7.4. The lungs of newborn mice were fixed by tracheal instillation of paraformaldehyde at a constant pressure of 20 cm H₂O. After overnight fixation, the lungs were dehydrated in graded ethanol solutions, embedded in paraffin, and stained with hematoxylin and eosin (H&E). Controls consisted of Dox-treated single-transgenic CCSP⁺/FasL⁻ littermates, and age-matched CCSP⁺/FasL⁺ and CCSP⁺/FasL⁻ animals that were not treated with Dox. All animal experiments were conducted in accordance with institutional guidelines for the care and use of laboratory animals. Protocols were approved through the Institutional Animal Care and Use Committee.

Alveolar Type II Cell Isolation and Culture

Alveolar type II cells were isolated from fetal mice (E19) by a modification of the methods described by Corti and colleagues⁴⁶ and Rice and colleagues,⁴⁷ as described in detail elsewhere.¹⁰ Briefly, type II cells were isolated by protease digestion and differential adherence to CD45- and CD32-coated dishes. After isolation and purification, the cells were resuspended in culture medium (HEPES-buffered Dulbecco's modified Eagle's medium, 10% fetal bovine serum, 100 U/ml penicillin, 100 μg/ml streptomycin). Purity was assessed by modified Papanicolaou stain⁴⁸ and anti-SP-C immunohistochemistry.¹⁰ Viability was assessed by trypan blue exclusion. After 24 hours, cells were assayed for FasL mRNA expression as detailed below.

Analysis of FasL Gene Expression

Quantitative Real-Time Polymerase Chain Reaction Analysis FasL mRNA levels were quantified by real-time PCR analysis. Total cellular RNA was extracted from whole lung or cell lysates using Trizol reagent (Invitrogen, Carlsbad, Calif.). Total RNA (2 μg) was DNase-treated (Turbo DNA-free kit; Ambion, Austin, Tex.) and reverse-transcribed using the reverse transcriptase² first strand kit (SuperArray BioScience) according to the manufacturer's protocols. The cDNA templates were amplified with mouse β-actin (Superarray catalog no. PPM02945A) and FasL (PPM02926A) primer pairs in independent sets of PCR using reverse transcriptase.² Real-time SYBR Green PCR master mix (Superarray) on an Eppendorf Mastercycler ep realplex (Westbury, N.Y.) according to the manufacturer's protocols. Each sample was run in triplicate, and mRNA levels were analyzed relative to the β-actin housekeeping gene. Relative gene expression ratios were calculated according to the SuperArray-recommended ΔΔCₜ protocol.⁴⁹

Immunohistochemical Analysis

The cellular distribution of FasL protein in lung tissues was studied by the streptavidin-biotin immunoperoxidase method using a polyclonal rabbit anti-FasL antibody (Chemicon/Millipore, Billerica, Mass.).^{10,11} Immunoreactivity was detected with 3,3′-diaminobenzidine tetrachloride. Specificity controls consisted of omission of primary antibody.

Analysis of Apoptosis

Terminal Deoxynucleotidyl Transferase-Mediated dUTP-FITC Nick-End (TUNEL) Labeling

Pulmonary apoptotic activity was localized and quantified by TUNEL labeling, as previously described.^{10,11} Negative controls for TUNEL labeling were performed by omission of the transferase enzyme. For quantification of TUNEL signals, a minimum of 25 high-power fields were viewed per sample, and the number of apoptotic nuclei per high-power field [apoptotic index (Al)] was recorded. To assess alveolar type II cell apoptosis, TUNEL labeling was combined with immunohistochemical detection of type II cells using a polyclonal anti-prosurfactant protein C (SP-C) antibody (Abcam Inc., Cambridge, Mass.), as described.^{11,12} To evaluate Clara cell apoptosis, TUNEL labeling was combined with immunohistochemical detection of Clara cells using a polyclonal anti-CCSP antibody (Upstate Biotechnology, Lake Placid, N.Y.). Negative controls included omission of the primary antibody, which abolished all staining.

Electron Microscopy

For ultrastructural studies, lung samples were fixed with 1.25% glutaraldehyde in 0.15 mol/L sodium cacodylate buffer, postfixed with 1% osmium tetroxide, and dehydrated through a graded ethanol series. Ultrathin sections were stained with uranyl acetate/lead citrate and viewed using a Philips 300 electron microscope (Philips, Research, Eindhoven, The Netherlands).

Western Blot Analysis of Caspase-3 Cleavage Processing and cleavage of the key Fas-dependent executioner caspase, caspase-3, was assayed by Western blot analysis of lung homogenates, as described elsewhere.^{10,22} Caspase-3 is expressed as an inactive 32-kDa precursor from which the 17-kDa and 20-kDa subunits of the mature caspase-3 are proteolytically generated during apoptosis. The rabbit polyclonal anticaspase-3 antibody used (Cell Signaling, Danvers, Mass.) detects the 17- and 20-kDa cleavage products generated during apoptosis as well as the 32-kDa precursor caspase. Secondary goat antibody was conjugated with horseradish peroxidase and blots developed with an enhanced chemiluminescence detection assay (Amersham Pharmacia Biotech, Piscataway, N.J.). Band intensity was expressed as the combined integrated optical density of the 17- and 20-kDa bands, normalized to the integrated optical density of actin bands (loading control). Specificity controls included preincubation of antibody with blocking peptide.

Analysis of Lung Growth, Alveolarization, and Microvascular Development

Lung growth was assessed by lung weight and lung weight/body weight ratio. In lungs processed for molecular analyses, wet lung weight was recorded. For morphological and morphometric studies, lungs were formalin-fixed by standardized tracheal instillation in situ and lung growth was assessed by determination of inflated lung weight. Morphometric assessment of growth of peripheral air-exchanging lung parenchyma and contribution of the various lung compartments (airspace versus parenchyma) to the total lung volume was performed using standard stereological volumetric techniques, as previously described.^{50,51} The inflated lung volume, V(lu), was determined according to the Archimedes principle.⁵² The areal density of air-exchanging parenchyma, AA(ae/lu), was determined by point-counting based on computer assisted image analysis. The number of points falling on air-exchanging parenchyma (peripheral lung parenchyma excluding airspace) in random lung fields was divided by the number of points falling on the entire field (tissue and airspace). AA(ae/lu) represents the tissue fraction of the lung and as such is the complement of the airspace fraction AA(air/lu). The total volume of air-exchanging parenchyma, V(ae), was calculated by multiplying AA(ae/lu) by V(lu). Alveolarization was quantified by computer-assisted morphometric analysis of the mean cord length (MCL) and radial alveolar count (RAC). MCLs were determined by superimposing randomly oriented parallel arrays of lines across randomly selected microscope fields of air-exchanging lung parenchyma (at least 25 random fields per lung) and determining the distance between airspace walls (including alveoli, alveolar sacs, and ducts). The MCL is an indirect estimate of the degree of airspace subdivision by alveolar septa.⁴¹ The RAC was determined by counting the number of septa intersected by a perpendicular line drawn from the center of a respiratory bronchiole to the edge of the acinus (connective tissue septum or pleura),⁵³ based on analysis of at least 10 randomly selected lung fields. The MCL was used to calculate mean volume of airspace units using the following formula: (MCL³×π)/3. The internal surface area of the lung available for gas exchange was calculated from the formula [(4×V(lu)]/MCL (adapted from Weibel and Cruz-Orive⁵⁴) and normalized to body weight to obtain the specific internal surface area. All morphometric assessments were made on coded slides from at least six animals per group by a single observer who was unaware of the genotype or experimental condition of the animal analyzed. For morphometric analysis of vessel density, sections were immunostained for the presence of Factor VIII [von Willebrand factor (vWF)] (DAKO, Carpinteria, Calif.), an endothelium-specific marker. The number of Factor VIII-positive vessels (20 to 80 μm in diameter) per high-power field (×20 objective) was counted in 25 randomly selected fields to assess the vessel density, as described by others.⁵⁵

Data Analysis

Values are expressed as mean±SD or, where appropriate, as mean±SEM. The significance of differences between groups was determined with the unpaired Student's t-test or analysis of variance with posthoc Scheffé test where indicated. The significance level was set at P<0.05. Statview software (Abacus, Berkeley, Calif.) was used for all statistical work.

Results

Generation of (tetOp)7-FasL Transgenic Mice

Pronuclear microinjection of the (tetOp)₇-FasL construct yielded 36 live pups that were screened by PCR. Five transgenic lines (A to E) were established successfully; the transgene copy number of these lines ranged from 2 to 35. The following studies are based on transgenic mouse line D, which has 20 transgene copies and showed intermediate levels of Dox-induced FasL mRNA up-regulation.

Dox Administration Induces Lung-Specific FasL Overexpression in Bitransgenic CCSP⁺/FasL⁺ Mice

Pulmonary FasL mRNA Expression

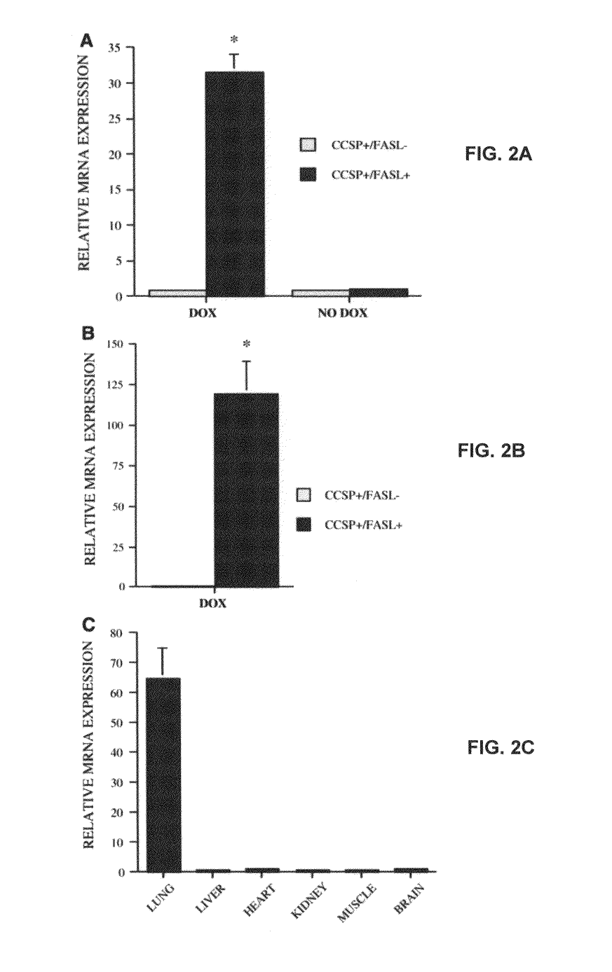

Transgenic (tetOp)⁷-FasL responder mice (male or female) were crossed with transgenic CCSP-rtTA activator mice of the opposite gender to obtain litters composed of double-transgenic CCSP⁺/FasL⁺ and single-transgenic CCSP⁺/FasL⁻ progeny. Dox was administered to pregnant, and subsequently, nursing dams from E14 to P7. The effect of Dox administration on FasL mRNA expression was studied by quantitative real-time PCR analysis of whole lung homogenates at P7. As shown in FIG. 2A, Dox treatment induced a robust more than 30-fold increase in pulmonary FasL mRNA levels in bitransgenic CCSP⁺/FasL⁺ mice compared with single-transgenic CCSP⁺/FasL⁻ littermates. In the absence of Dox, the FasL mRNA levels of double-transgenic littermates were similar to those of single-transgenic littermates (FIG. 2A), indicating that there was no Dox-independent transgene leak.

To verify that the CCSP promoter construct is effective in inducing transgene expression in alveolar type II cells, FasL mRNA levels were determined in primary alveolar type II cells isolated from Dox-treated CCSP⁺/FasL⁺ and CCSP⁺/FasL⁻ mice at E19. As seen in FIG. 2B, the FasL mRNA levels were significantly more than 100-fold higher in type II cells from Dox-treated double-transgenic CCSP⁺/FasL⁺ mice compared with single-transgenic littermates. This confirms that the CCSP promoter construct in CCSP-rtTA mice is capable of driving transgene expression in alveolar type II cells, unlike the endogenous murine Clara cell-restricted CCSP promoter. The lung specificity of Dox-induced FasL transgene expression was determined by real-time PCR analysis of FasL mRNA levels in a variety of organs and tissues. As shown in FIG. 2C, Dox-induced FasL up-regulation in CCSP⁺/FasL⁺ mice was limited to the lung, and not seen in liver, heart, kidneys, muscle, or brain.

FasL Immunohistochemistry

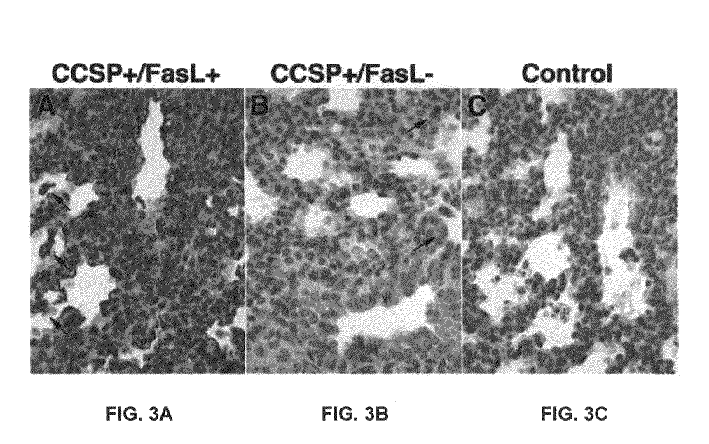

The cellular distribution of FasL protein was determined by immunohistochemical analysis (FIG. 3). Lungs of Dox-treated double-transgenic CCSP⁺/FasL⁺ mice (E19) showed intense and diffuse FasL immunoreactivity, localized to bronchial epithelial cells, alveolar epithelial cells, and intra-alveolar cellular debris (FIG. 3A). In single-transgenic CCSP⁺/FasL littermates, FasL immunostaining was modest and localized to bronchial epithelial cells and scattered alveolar epithelial cells that were morphologically consistent with alveolar type II cells (FIG. 3B). Omission of the primary antibody abolished all immunoreactivity (FIG. 3C).

FasL Overexpression in Dox-Treated Bitransgenic CCSP⁺/FasL⁺ Mice Is Associated with Increased Postnatal, but Not Fetal Lethality

To assess the effects of FasL transgene overexpression on antenatal and postnatal viability, the proportions of double-transgenic CCSP⁺/FasL⁺ and single-transgenic CCSP⁺/FasL⁻ progeny at E19 and at P7 were determined. CCSP-rtTA mice are homozygous and (TetOp)₇-FasL mice hemizygous for their respective transgenes. According to Mendelian laws of inheritance, equal proportions of double-transgenic CCSP⁺/FasL⁺ and single-transgenic CCSP⁺/FasL⁻ progeny would be expected in the absence of a lethal effect.

At E19 (late gestation), the ratios of double- and single-transgenic Dox-treated fetuses were approximately equal, indicating that the CCSP⁺/FasL⁺ double-transgenic status does not confer lethal effects in utero (Table 4). At P7, however, CCSP⁺/FasL⁺ pups accounted for only 30% of Dox-treated progeny, suggestive of increased postnatal lethality in Dox-exposed double-transgenic mice (Table 5). The body weight of Dox-treated double-transgenic mice at P7 tended to be less than that of single-transgenic littermates, whereas their lung weight tended to be larger (Table 5). This resulted in significantly larger lung weight/body weight ratios in Doxtreated double-transgenic mice compared with single-transgenic littermates. In the absence of Dox, the ratios of double- and single-transgenic progeny at P7 were equal, indicating that the double-transgenic CCSP⁺/FasL⁺ status is not deleterious without Dox-exposure. The body weights of Dox-treated transgenic animals were significantly lower than those of non-Dox-treated animals of the same genotype, suggesting that Dox affects postnatal somatic growth (Table 5). The body weights of Doxtreated or non-Dox-treated pups were not affected by the maternal genotype (CCSP-rtTA versus (tetOp)₇-FasL).

TABLE 4
Genetic and Biometric Characteristics of Dox-Treated
Transgenic CCSP/FasL Mice at E19

Age E19

| Genotype | CCSP⁺/FasL⁺ | CCSP⁺/FasL⁻ |
|---|---|---|
| Fraction of | 24/51 (47%) | 27/51 (53%) |
| living | | |
| offspring | | |
| Body weight | 1.11 ± 0.11 (24) | 1.11 ± 0.11 (27) |
| (g) | | |
| Lung weight | 0.023 ± 0.006 (17) | 0.026 ± 0.006 (21) |
| (g) | | |
| Lung weight/ | 2.16 ± 0.54 (17) | 2.42 ± 0.01 (21)* |
| body | | |
| weight (%) | | |

Lung weights at E19 reflect wet lung weights. Values represent mean ± SD.
*P < 0.05 versus CCSP⁺/FasL⁺ littermates (Student's t-test).

TABLE 5
Genetic and Biometric Characteristics of Dox-Treated or Non-Dox-Treated
Transgenic CCSP/FasL Mice at P7

| | Age P7 | | | |
|---|---|---|---|---|
| | Dox | | No Dox | |
| | CCSP⁺/FasL⁺ | CCSP⁺/FasL⁻ | CCSP⁺/FasL⁺ | CCSP⁺/FasL⁻ |
| Fraction of living offspring | 22/72 (31%) | 50/72 (69%) | 8/16 (50%) | 8/16 (50%) |
| Body weight (g) | 2.78 ± 0.48 (22) | 2.99 ± 0.47 (50) | 3.66 ± 0.27 (8)^{‡} | 3.65 ± 0.17 (8)^{‡} |
| Lung weight (wet) (g) | 0.068 ± 0.009 (5) | 0.062 ± 0.004 (8) | 0.057 ± 0.002 (4) | 0.057 ± 0.008 (4) |
| Lung weight (wet)/body weight (%) | 2.17 ± 0.45 (5) | 1.76 ± 0.21 (8)* | 1.63 ± 0.15 (4) | 1.49 ± 0.18 (4) |
| Lung weight (infl) (g) | 0.21 ± 0.03 (5) | 0.19 ± 0.03 (11) | 0.18 ± 0.01 (3) | 0.18 ± 0.01 (3) |
| Lung weight (infl)/body weight (%) | 7.32 ± 0.65 (5) | 6.15 ± 0.73 (11)^{†} | 4.98 ± 0.09 (3)^{‡} | 4.77 ± 0.25 (3)^{‡} |

Values represent mean ± SD.
*P < 0.05 versus CCSP⁺/FasL⁺ littermates;
^{†}P < 0.01 versus CCSP⁺/FasL⁺ littermates;
^{‡}P < 0.01 versus Dox-treated animals of same genotype (Student's t-test).

FasL Overexpression in Dox-Treated Bitransgenic CCSP⁺/FasL⁺ Mice

Induces Increased Pulmonary Apoptosis

Lung Morphology and TUNEL Analysis at E19 and P7

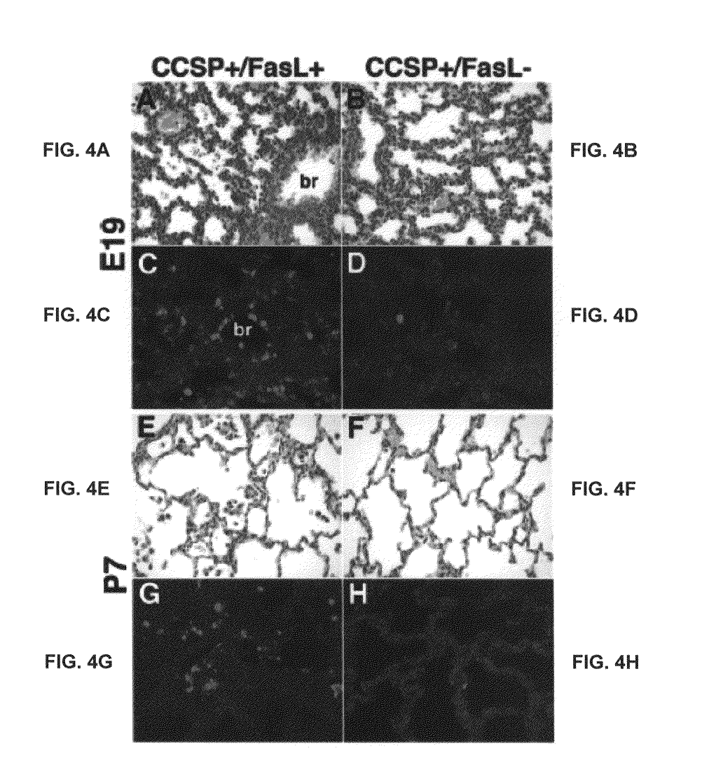

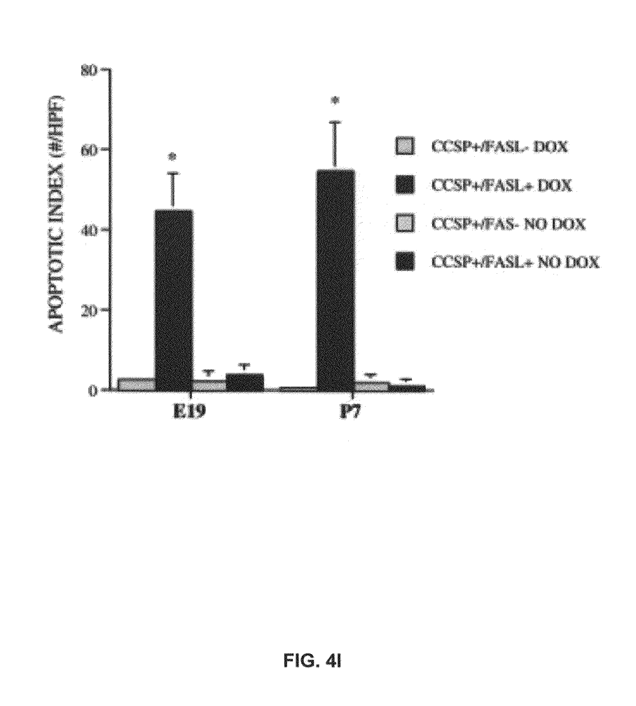

Lungs of double-transgenic CCSP⁺/FasL⁺ mice treated with Dox from E14 on and examined at E19 and P7 showed abundant cellular debris and detached apoptotic cells within the airspaces (FIG. 4, A and E). At P7, the intra-alveolar cellular material was admixed with numerous macrophages. Pyknotic nuclei within the bronchial epithelium were readily observed at both time points (FIG. 4, A and E). The lungs of single-transgenic CCSP⁺/FasL⁻ littermates appeared unremarkable and showed only rare single cells in the airspaces (FIG. 4, B and F).

TUNEL labeling highlighted the dramatic increase in apoptotic activity in lungs of Dox-treated double-transgenic mice (FIG. 4, C and G). The increased TUNEL activity in Dox-exposed double-transgenic mice was noted in cellular debris within the airspaces and in bronchial epithelial cells. The apoptotic activity in single-transgenic lungs was low at both E19 and P7, and mainly localized to peribronchial and perivascular stromal cells (FIG. 4, D and H). In the absence of Dox, the apoptotic activity of CCSP⁺/FasL⁺ mice was very low and similar to that of single-transgenic littermates (FIG. 41).

Interval studies determined that virtually all (>95%) Dox-exposed mice that died between birth and P7 had the double-transgenic CCSP⁺/FasL⁺ genotype. Histopathological studies of the lungs of these animals showed that their airspaces were massively occluded by cellular debris admixed with apoptotic nuclear material (not shown). Based on this pathological evidence and the gross appearance of the moribund pups, the disproportionate postnatal lethality of Dox-treated CCSP⁺/FasL⁺ mice was attributed to respiratory failure.

TUNEL Analysis Combined with Anti-SP and Anti-CCSP Immunolabeling at E19 and P7

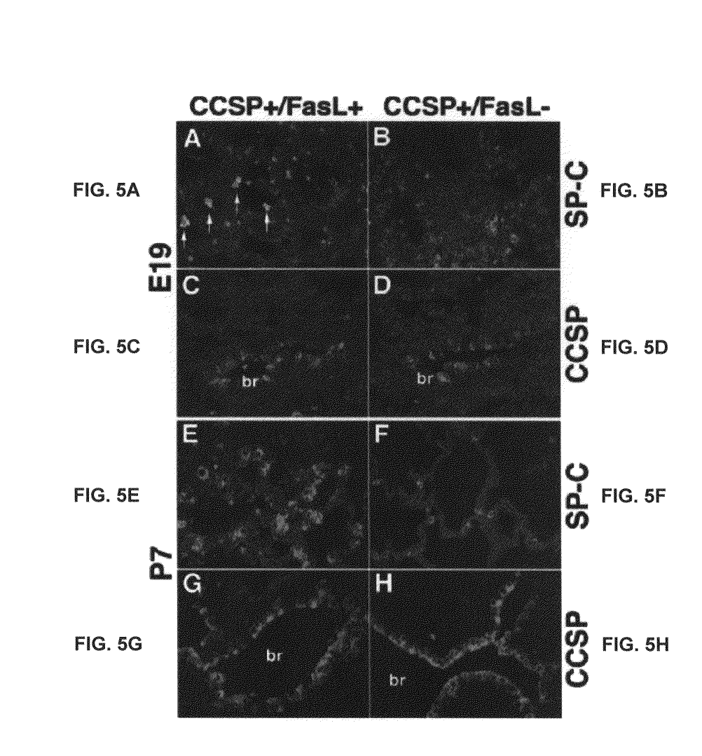

To determine the identity of the apoptotic cells, TUNEL labeling was combined with anti-SP-C or anti-CCSP immunohistochemistry. At E19, lungs of Dox-treated double-transgenic CCSP⁺/FasL⁺ mice showed frequent association of TUNEL-positive nuclei with SP-C immunoreactive cellular material within the intra-alveolar debris, suggesting that a large proportion of apoptotic cells were type II cells (FIG. 5A). In single-transgenic littermates, the scant pulmonary apoptotic activity was mainly seen in SP-C-negative peribronchial and perivascular stromal cells (FIG. 5B). Combination of TUNEL labeling with anti-CCSP staining revealed brisk apoptotic activity in CCSP-positive bronchial epithelial Clara cells in double transgenic CCSP⁺/FasL⁺ mice (FIG. 5C), whereas apoptotic activity of Clara cells was negligible in single transgenic mice (FIG. 5D).

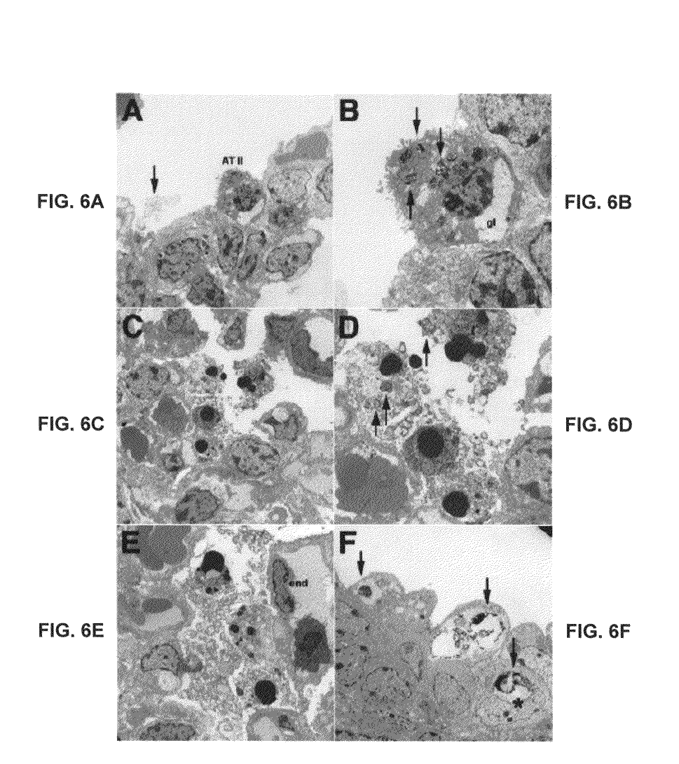

At P7, abundant apoptotic cellular debris remained present within the airspaces of Dox-treated CCSP⁺/FasL⁺ mice (FIG. 5E). At this time point, most intraalveolar apoptotic nuclei were devoid of identifiable cellular characteristics. Strikingly, lungs of CCSP⁺/FasL⁺ mice at P7 showed large numbers of intensely SP-C immunoreactive alveolar type II cells within the alveolar walls. As seen in FIG. 6E, these hyperplastic type II cells were virtually uniformly TUNEL-negative.

TUNEL labeling combined with anti-CCSP immunostaining at P7 demonstrated increased numbers of TUNEL-positive nuclei in the bronchial epithelium of double-transgenic CCSP⁺/FasL⁺ mice, both in CCSP-positive Clara cells and in neighboring CCSP-negative bronchial epithelial cells (FIG. 5G). The apoptotic activity in Dox-treated single-transgenic CCSP⁺/FasL⁻ littermates was low, and preferentially localized to SP-C-negative and CCSP-negative stromal cells around large-sized vascular or bronchial structures (FIGS. 5F and H).

Electron Microscopy

Ultrastructural analysis of the lungs of Dox-treated single-transgenic CCSP⁺/FasL⁻ mice (E19) revealed frequent, well preserved alveolar type II cells that were readily identified by their cuboidal shape, prominent microvilli, and the presence of cytoplasmic lamellar bodies and glycogen pools (FIG. 6, A and B). The alveolar space overlying the alveolar type II cells often showed the presence of tubular myelin, consistent with surfactant lipid material.

In contrast, the lungs of Dox-treated double-transgenic CCSP⁺/FasL⁺ littermates showed a striking paucity of recognizable alveolar type II cells. Instead, these lungs contained large aggregates of detached cells within the airspaces that were often associated with tubular myelin-like material. These detached intra-alveolar cells showed the characteristic ultrastructural features of apoptosis, including cell shrinkage and peripheral or diffuse chromatin condensation of the nuclei (FIG. 6, C-E). Definitive identification of the detached apoptotic cells was often impossible because of advanced cellular degradation. In better preserved apoptotic cells, however, typical cytoplasmic lamellar bodies could be seen, allowing identification of these cells as alveolar type II cells (FIG. 6D). Apoptotic nuclei with typical peripheral chromatin condensation were also seen in nonciliated bronchial epithelial (Clara) cells (FIG. 6F). Apoptotic nuclei were occasionally present in adjacent nonapoptotic bronchial epithelial cells, suggestive of phagocytotic clearance of apoptotic Clara cells by neighboring cells. Apoptotic nuclei were not observed in other bronchial epithelial cells, endothelial cells, or interstitial stromal cells.

Western Blot Analysis of Caspase-3 Cleavage

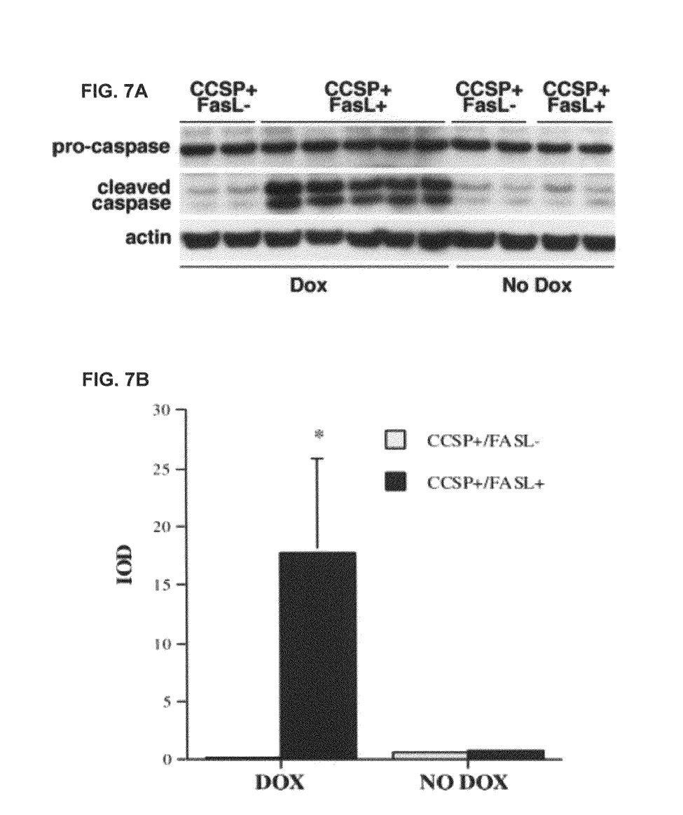

Processing and cleavage of caspase-3, the main executioner of the Fas-dependent apoptotic machinery, was assessed by Western blot analysis using an antibody specific for both procaspase-3 and the active caspase-3 cleavage products. Consistent with Fas-mediated cell death, Dox-exposed CCSP⁺/FasL⁺ lungs showed cleavage of procaspase-3 and increased levels of the immunoreactive 17- and 20-kDa active subunits of caspase-3 (FIG. 7). In contrast, levels of the caspase split products were negligible in lung homogenates of CCSP⁺/FasL⁻ littermates. In the absence of Dox, caspase-3 cleavage was minimal and similar in double- and single-transgenic mice.

FasL⁻Induced Alveolar Epithelial Apoptosis Disrupts Alveolar and Microvascular Development in Dox-Treated Bitransgenic CCSP⁺/FasL⁺ Mice

To determine the effects of FasL-induced apoptosis on alveolar remodeling, lungs of transgenic mice were studied at P21 after Dox administration from E14 to P7. The ratios of surviving Dox-treated double- and single-transgenic progeny at P21 (29% double transgenic, 71% single transgenic) were similar to those observed at P7, suggesting that no additional mortality occurred after discontinuation of the Dox treatment (Table 6). By P21, the body weights were similar in Dox-treated double- and single-transgenic animals and equivalent to those of animals not exposed to Dox (Table 6). The lung weight/body weight ratio (either wet or inflation-fixed) was significantly larger in Dox-treated CCSP⁺/FasL⁺ mice than in CCSP⁺/FasL⁻ littermates (P<0.01) (Table 6).

TABLE 6
Genetic and Biometric Characteristics of Dox-Treated or Non-Dox-Treated
Transgenic CCSP/FasL Mice at P21

| | Age P21 | | | |
|---|---|---|---|---|
| | Dox | | No Dox | |
| | CCSP⁺/FasL⁺ | CCSP⁺/FasL⁻ | CCSP⁺FasL⁺ | CCSP⁺/FasL⁻ |
| Fraction of living offspring | 10/35 (29%) | 25/35 (71%) | 7/13 (54%) | 6/13 (46%) |
| Body weight (g) | 10.25 ± 0.96 (10) | 10.96 ± 1.45 (25) | 10.16 ± 0.58 (7) | 9.85 ± 0.24 (6) |
| Lung weight (wet) (g) | 0.12 ± 0.01 (3) | 0.11 ± 0.01 (7) | 0.12 ± 0.01 (4) | 0.11 ± 0.01 (3) |
| Lung weight (wet)/body weight (%) | 1.36 ± 0.06 (3) | 1.21 ± 0.05 (7)* | 1.14 ± 0.15 (4) | 1.14 ± 0.13 (3) |
| Lung weight (infl) (g) | 0.50 ± 0.05 (6) | 0.43 ± 0.03 (16) | 0.39 ± 0.03 (3)^{†} | 0.35 ± 0.01 (3)^{†} |
| Lung weight (infl)/body weight (%) | 4.68 ± 0.48 (6) | 3.78 ± 0.42 (16)* | 3.95 ± 0.18 (3)^{‡} | 3.57 ± 0.05 (3) |

Values represent mean ± SD.
Lung weight (infl): lung weight after standardized inflation and fixation.
*P < 0.01 versus CCSP⁺/FasL⁺ littermates;
^{†}P < 0.01 versus Dox-treated animals of same genotype;
^{‡}P < 0.05 versus Dox-treated animals of same genotype (Student's t-test).

The total lung volume, V(lu), and the V(lu)/body weight ratio were significantly larger in Dox-treated double-transgenic mice than in single-transgenic littermates (P<0.01) (Table 7). Computer-assisted stereological volumetry was applied to determine the relative contributions of the various lung compartments (specifically, peripheral air-exchanging parenchyma versus airspace) to the observed total lung volume. The areal density of air-exchanging parenchyma, A_{A}(ae/lu), representing the parenchymal tissue fraction, was significantly lower in double-transgenic mice. The total volume of air-exchanging parenchyma, V(ae), which takes into account both A_{A}(ae/lu) and V(lu), was similar in double- and single-transgenic mice, indicating that the increased V(lu) in double-transgenic mice was attributable to distension of the airspaces rather than actual tissue growth.

TABLE 7
Morphometric Analysis of Lungs of Dox-Treated or Non-Dox-Treated
Transgenic CCSP/FasL Mice at P21

| | Age P21 | | | |
|---|---|---|---|---|
| | Dox | | No Dox | |
| | CCSP⁺/FasL⁺ | CCSP⁺/FasL⁻ | CCSP⁺/FasL⁺ | CCSP⁺/FasL⁻ |
| V(lu) (μl) | 471 ± 52 (6) | 407 ± 30 (16)* | 372 ± 26 (3)^{‡} | 334 ± 8 (3)^{‡} |
| V(lu)/body weight (ml/g) | 4.44 ± 0.46 (6) | 3.59 ± 0.40 (16)* | 3.75 ± 0.20 (3)^{†} | 3.39 ± 0.05 (3) |
| A_{A}(ae/lu) (%) | 28.06 ± 2.51 (6) | 33.22 ± 3.17 (16)* | 32.04 ± 1.74 (3)^{†} | 33.71 ± 1.13 (3) |
| A_{A}(air/lu) (%) | 71.94 ± 2.51 (6) | 66.78 ± 3.17 (16)* | 67.96 ± 1.74 (3)^{†} | 66.29 ± 1.13 (3) |
| V(ae) (μl) | 131 ± 9 (6) | 135 ± 18 (16) | 119 ± 9 (3) | 113 ± 6 (3) |
| V(aspunit) (μl × 10⁻⁶) | 42.9 ± 8.8 (6) | 15.5 ± 4.1 (16)* | 6.6 ± 0.9 (3)^{‡} | 7.1 ± 1.2 (3)^{‡} |
| ISA (cm²) | 553 ± 90 (6) | 685 ± 66 (16)* | 808 ± 68 (3)^{‡} | 711 ± 55 (3) |
| SISA (cm²/g) | 52.2 ± 8.7 (6) | 59.7 ± 8.3 (16) | 81.5 ± 7.2 (3)^{‡} | 72.0 ± 5.0 (3)^{†} |

Values represent mean ± SD. V(lu), lung volume;
A_{A}(ae/lu), fraction of air-exchanging parenchyma;
A_{A}(air/lu), airspace fraction;
V(ae), volume of air-exchanging parenchyma;
V(aspunit), volume of airspace unit;
ISA: internal surface area;
SISA, specific internal surface area.
*P < 0.01 versus CCSP⁺/FasL⁺ littermates;
^{†}P < 0.05 versus Dox-treated animals of same genotype;
^{‡}P < 0.01 versus Dox-treated animals of same genotype (Student's t-test).

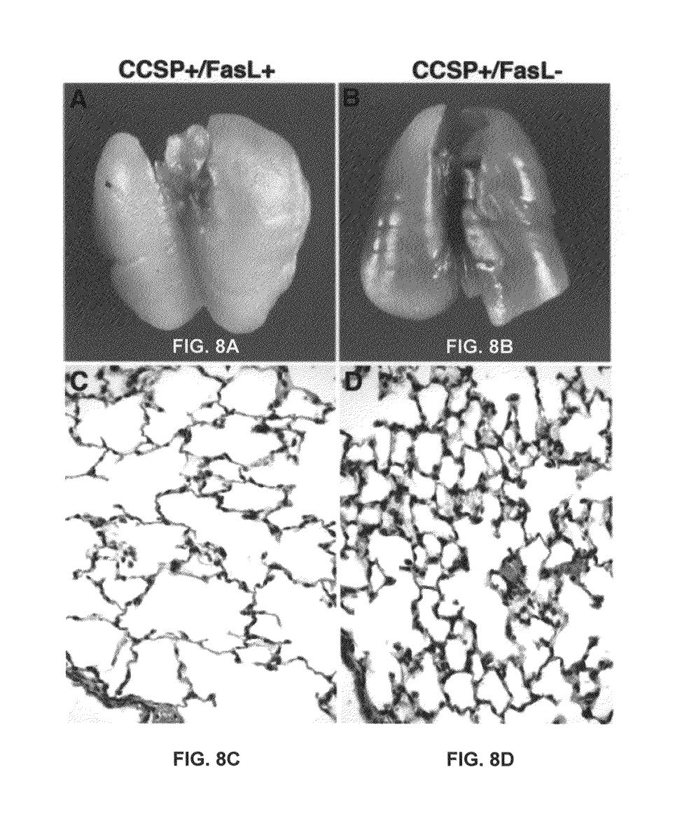

The formalin-inflated lungs of Dox-treated double-transgenic CCSP⁺/FasL⁺ mice at P21 appeared large and pale compared with those of CCSP⁺/FasL⁻ littermates (FIG. 8, A and B). Microscopically, lungs of Dox-treated CCSP⁺/FasL⁺ mice displayed marked alveolar disruption characterized by large-sized simplified airspaces with a striking paucity of alveolar septation or secondary crest formation, thus faithfully mimicking the alveolar pathology of human BPD (FIG. 8C). Small macrophage collections were occasionally noted. There was no histopathological evidence of fibrosis or bronchial epithelial injury. Lungs of Dox-treated single-transgenic mice at P21 showed more advanced alveolarization, characterized by a more complex network of abundant small-sized polygonal alveoli, separated by delicate alveolar septa (FIG. 8D). Although more complex than in Dox-treated double-transgenic mice, alveolarization appeared less advanced in Dox-treated single-transgenic mice compared with non-Dox-treated transgenic mice (not shown).

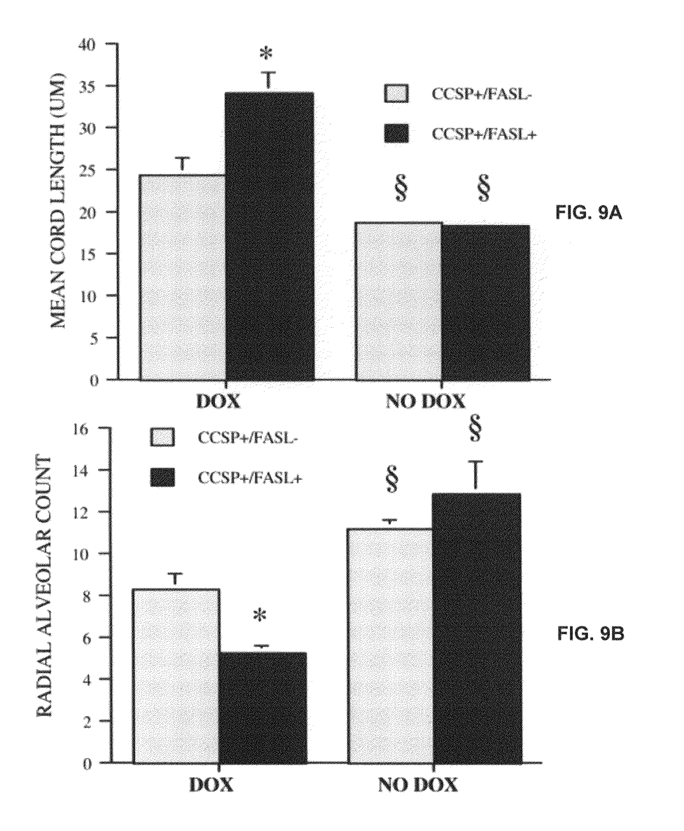

To estimate the degree of alveolarization in transgenic mice, the MCL and RAC were determined. As shown in FIG. 9, the MCL was significantly larger (P<0.01), and the RAC significantly smaller (P<0.01) in Dox-treated double-transgenic mice compared with single-transgenic littermates, confirming the presence of decreased alveolar septation in Dox-treated CCSP⁺/FasL⁺ mice. Without Dox, the MCL and RAC of single- and double-transgenic animals were similar (FIG. 9). The calculated volume of the individual airspace units was almost threefold larger in Dox-treated double-transgenic mice compared with single-transgenic mice (P<0.01) (Table 7). The lung surface area available for gas exchange, estimated by calculation of the internal surface area, was significantly diminished in Dox-treated CCSP⁺/FasL⁺ mice compared with single-transgenic littermates, although this difference was attenuated after normalization to body weight (Table 7).

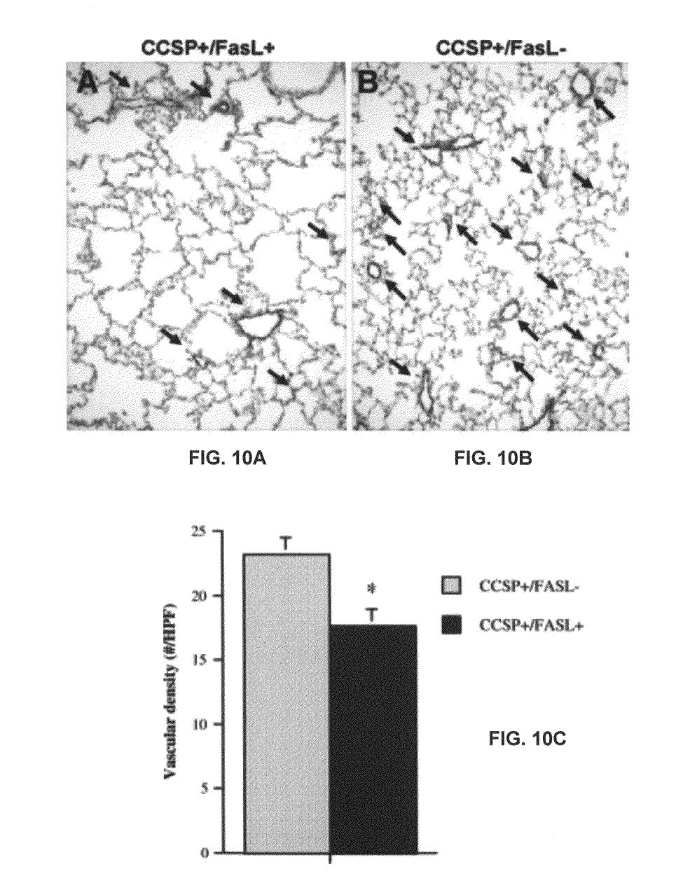

In the absence of Dox, the morphometric assessment of lung growth and alveolarization was similar in double and single-transgenic animals, indicating that the presence of the (TetOp)₇-FasL transgene does not have Dox-independent effects on lung development. Compared with non-Dox-treated single-transgenic CCSP⁺/FasL⁻ mice, Dox-treated single-transgenic animals had significantly larger V(lu), MCL, volume of airspace unit, and specific internal surface area, as well as significantly smaller RAC, indicative of Dox-related effects on alveolarization (FIG. 9 and Table 7). To assess the effect of FasL-mediated respiratory epithelial apoptosis on pulmonary microvascular development, the vascular density was determined, as described by others.⁵⁵ Vessel density was reduced by 25% in Dox-treated double-transgenic CCSP⁺/FasL⁺ mice compared with single-transgenic littermates (FIG. 10).

Discussion

In this study, a gain-of-function approach was used to determine the effects of alveolar epithelial apoptosis on postcanalicular alveolar remodeling. A tetracycline-inducible lung epithelial-specific FasL-overexpressing mouse was generated, adapted from the Tet system of Gossen and colleagues,³⁴ to target apoptosis to respiratory epithelial cells during perinatal lung development. Increased alveolar epithelial apoptosis during postcanalicular lung remodeling was determined to be sufficient to disrupt alveolar development and results in a pattern of alveolar simplification that closely mimics the pulmonary pathology of human BPD.

These findings establish a solid causative relationship between alveolar epithelial apoptosis and disrupted alveolarization and support the hypothesis that excessive or premature alveolar epithelial apoptosis is a pivotal event in the pathogenesis of BPD that links the known risk factors of BPD to the final common outcome: impaired alveolar development. Accumulating clinical and experimental evidence shows that the major predisposing factors implicated in BPD, including hyperoxia/oxygen toxicity,^{22,56,57} mechanical distension (stretch),^{12,58-61} and proinflammatory factors^{26,62} are capable of inducing alveolar epithelial apoptosis. The molecular signaling pathways regulating alveolar epithelial apoptosis in early BPD remain undetermined, but likely include both receptor-mediated (extrinsic) and mitochondrial-dependent (intrinsic) death signaling systems.

The precise mechanisms by which exaggerated or premature alveolar epithelial apoptosis induces alveolar disruption remain to be determined. First, the alveolar arrest may simply be attributable to numerical loss of alveolar type II cells during crucial time points of alveolar remodeling. Alveolar type II cells ensure adequate surfactant production around birth and serve as the proliferative source for alveolar type I cells that line most of the alveolar surface.⁶³ It is therefore reasonable to speculate that accumulation of a critical mass of alveolar type II cells is essential for normal postnatal lung remodeling. Interestingly, several lines of evidence in humans and experimental models link apoptosis with lung destruction in emphysematous adult lungs,⁶⁴⁻⁷⁰ suggesting that in fully developed lungs as well, type II cell loss is capable of disrupting the alveolar architecture.

Second, reactive type II cell hyperplasia after initial type II cell apoptosis may contribute, paradoxically, to disrupted alveolar remodeling. Reactive type II cell hyperplasia is a characteristic feature of most forms of acute lung injury, including the early stages of lung injury in newborns. In the present study, FasL overexpression in fetal lungs caused massive apoptosis of alveolar type II cells, resulting in near-total eradication of these cells by E19. By P7, however, the alveoli were repopulated by large numbers of strongly SP-C-immunoreactive alveolar type II cells. This newly emerging population of hyperplastic alveolar type II cells was strikingly refractory to apoptosis, despite continued Dox exposure and pulmonary FasL overexpression. This suggests that, at least with respect to Fas sensitivity, the second-generation type II cells are phenotypically different from the original, naïve type II cells. The cellular ontogeny and phenotypic characteristics of the repopulating type II cells remain to be determined. It is possible, however, that the hyperplastic type II cells may also differ from naïve type II cells in other aspects affecting alveologenesis, such as the epithelial-mesenchymal and epithelial-endothelial interactions required for alveolar septation.

Finally, apoptosis-induced alveolar disruption may be mediated by the action of macrophages and other proinflammatory mediators. Lungs of Dox-treated CCSP⁺/FasL⁺ mice at P7 contained large numbers of intra-alveolar macrophages, admixed with apoptotic cellular debris. Similarly, macrophages, neutrophils, and associated proinflammatory mediators are a constant feature in BPD.⁵ This BPD-associated inflammatory response, attributed to antenatal chorioamnionitis and intrauterine cytokine expression, as well as postnatal lung injury caused by resuscitation, oxygen toxicity, volutrauma, barotraumas, and infection, has been implicated in inhibition of alveolarization in the lungs of preterm infants.^{71,72} This view of BPD may need to be integrated with the angiocentric paradigm emphasized in the current literature.⁷³ It has been shown that, in addition to impaired alveolar development, there is also a disruption of pulmonary microvascular development in infants with BPD^{5,6,74} or in BPD-like animal models such as chronically ventilated premature baboons.^{75,76} Although there is controversy whether angiogenesis is increased⁶ or decreased,⁷³ there is general agreement that the microvasculature is dysmorphic in BPD.^{5,6,73,74} In the present study, the pulmonary vessel density was significantly lower in Dox-treated double-transgenic mice compared with single-transgenic littermates, similar to the microvascular anomalies seen in infants with BPD.

Several observations require special consideration. First, the clearance mechanisms for apoptotic alveolar type II cells and apoptotic Clara cells were found to be strikingly different. Alveolar type II cell apoptosis resulted in massive detachment of these cells from the alveolar wall and subsequent phagocytosis by intra-alveolar macrophages. The vast majority of apoptotic Clara cells, in contrast, remained attached to the bronchial epithelial wall or underwent phagocytosis by adjacent bronchial epithelial cells. The exact mechanisms underlying differential clearance mechanisms in various apoptotic respiratory epithelial cells remain unclear, but are likely related to the nature and extent of lateral cell-cell interactions.

Second, the apoptotic effects of FasL overexpression were virtually limited to alveolar type II cells and bronchial epithelial Clara cells. FasL up-regulation in Dox-treated CCSP⁺/FasL⁺ mice did not induce noticeable apoptosis in non-Clara bronchial epithelial cells, interstitial stromal cells, fibroblasts, or endothelial cells. The resistance of non-Clara bronchial epithelial cells to FasL activation was particularly striking because bronchial epithelial cells strongly express Fas receptor.^{10,11,77-79} The refractoriness of airway epithelial cells to Fas-induced apoptosis has been reported previously⁶² and has been ascribed to the expression of prosurvival proteins such as the caspase inhibitors c-IAP1 and c-IAP-2.^{80,81}

Use of a tetracycline-regulated bitransgenic expression system in vivo requires rigorous controls to ensure accurate interpretation of the data.⁸² Potentially confounding variables that may influence the outcome include integration site effects (such as insertional mutagenesis), copy number effects, the effects of Dox exposure, and potential toxicity of rtTA transgene. Five (TetOp)7-FasL transgenic lines were successfully established. The number of transgene copies in these lines ranged from 2 to 30. When crossed with CCSP-rtTA animals, the up-regulation of pulmonary FasL mRNA ranged from 5-fold to more than 1000-fold. The present study was focused on a transgenic line with intermediate transgene copy numbers (20) and intermediate levels of FasL up-regulation (30-fold). All lines, however, showed similar phenotypical features ascribed to FasL overexpression (i.e., increased pulmonary apoptosis and arrested alveolar development), and the severity of their phenotype correlated with the level of FasL mRNA up-regulation. The occurrence of apoptosis and alveolar disruption in all transgenic lines studied indicates that the pulmonary phenotype described in this study is a specific effect of transgene expression, and not a result of nonspecific integration or copy number effects.

The tetracycline-regulated expression system uses the tetracycline analogue, Dox, for induction of transgene expression. Two distinct Dox-related phenotypic effects were identified in the present study. As previously described by others in postnatal rats,⁸³ Dox treatment during the perinatal period was found to have adverse effects on early postnatal somatic growth. Whether the lower body weight of Dox-treated pups was the result of indirect effects on the nursing dams or direct effects on growth of the pups remains undetermined. In accordance with previous reports,⁸³ Dox was further found to negatively affect alveolar development, a phenomenon that has been attributed to its various nonantibiotic activities that include pan-MMP (matrix metalloproteinase) inhibition^{83,84} and antiangiogenic and anti-inflammatory effects.⁸⁵ Importantly, this study was controlled for Dox-related effects by comparing Dox-treated double-transgenic CCSP⁺/FasL⁺ animals with Dox-treated CCSP⁺/FasL⁻ littermates.

In summary, a transgenic mouse that allows external control of FasL expression in the respiratory epithelium was generated. Pulmonary FasL overexpression targeted to the postcanalicular stages of lung development was demonstrated to be sufficient to induce alveolar epithelial apoptosis and arrested alveolar development, mimicking the pulmonary pathology of BPD. These results support the hypothesis that alveolar epithelial apoptosis is a pivotal event in the pathogenesis of BPD. The versatility of the novel tetracycline-inducible CCSP/FasL mouse model should facilitate the analysis of the pathogenesis and new therapeutic approaches for BPD and other perinatal or adult pulmonary diseases characterized by dysregulated alveolar epithelial apoptosis. Furthermore, (TetOp)7-FasL transgenic mice, cross-bred with mice carrying the appropriate cell-specific rtTA or tTA construct, may be invaluable models to study the effects of Fas-mediated apoptosis in a wide range of conditions and organ systems.

### REFERENCES

1. Jobe A H, Bancalari E: Bronchopulmonary dysplasia. Am J Respir Crit Care Med 2001, 163:1723-1729
2. Lemons J A, Bauer C R, Oh W, Korones S B, Papile L A, Stoll B J, Verter J, Temprosa M, Wright L L, Ehrenkranz R A, Fanaroff A A, Stark A, Carlo W, Tyson J E, Donovan E F, Shankaran S, Stevenson D K: Very low birthweight outcomes of the National Institute of Child health and human development neonatal research network, January 1995 through December 1996 NICHD Neonatal Research Network. Pediatrics 2001, 107:E1
3. Husain A N, Siddiqui N H, Stocker J T: Pathology of arrested acinar development in postsurfactant bronchopulmonary dysplasia. HumPathol 1998, 29:710-717
4. Jobe A J: The new BPD: an arrest of lung development. Pediatr Res1999, 46:641-643
5. Coalson J J: Pathology of chronic lung disease in early infancy Chronic Lung Disease in Early Infancy. Edited by Bland R D, Coalson J J. New York, M. Dekker, 2000, pp 85-124
6. De Paepe M E, Mao Q, Powell J, Rubin S E, DeKoninck P, Appel N, Dixon M, Gundogan F: Growth of pulmonary microvasculature inventilated preterm infants. Am J Respir Crit Care Med 2006, 173:204-211
7. Northway W H Jr, Rosan R C, Porter D Y: Pulmonary disease followingrespirator therapy of hyaline-membrane disease. Bronchopulmonarydysplasia. N Engl J Med 1967, 276:357-368
8. Palta M, Gabbert D, Weinstein M R, Peters M E: Multivariate assessment of traditional risk factors for chronic lung disease in very lowbirth weight neonates. The Newborn Lung Project. J Pediatr 1991, 119:285-292
9. Jobe A H: Antenatal factors and the development of bronchopulmonary dysplasia. Semin Neonatol 2003, 8:9-17
10. De Paepe M E, Mao Q, Embree-Ku M, Rubin L P, Luks F I: Fas/FasLmediated apoptosis in perinatal murine lungs. Am J Physiol 2004, 287:L730-L742
11. De Paepe M E, Rubin L P, Jude C, Lesieur-Brooks A M, Mills D R, Luks F I: Fas ligand expression coincides with alveolar cell apoptosis in late-gestation fetal lung development. Am J Physiol 2000, 279:L967-L976
12. De Paepe M E, Sardesai M P, Johnson B D, Lesieur-Brooks A M, Papadakis K, Luks F I: The role of apoptosis in normal and accelerated lung development in fetal rabbits. J Pediatr Surg 1999, 34:863-870
13. Kresch M J, Christian C, Wu F, Hussain N: Ontogeny of apoptosis during lung development. Pediatr Res 1998, 43 :426-431
14. Schittny J C, Djonov V, Fine A, Burri P H: Programmed cell death contributes to postnatal lung development. Am J Respir Cell Mol Biol 1998, 18:786-793
15. Hargitai B, Szabo V, Hajdu J, Harmath A, Pataki M, Farid P, Papp Z, Szende B: Apoptosis in various organs of preterm infants: histopathologic study of lung, kidney, liver, and brain of ventilated infants. Pediatr Res 2001, 50:110-114
16. Lukkarinen H P, Laine J, Kaapa P O: Lung epithelial cells undergo apoptosis in neonatal respiratory distress syndrome. Pediatr Res 2003, 53:254-259
17. May M, Strobel P, Preisshofen T, Seidenspinner S, Marx A, Speer CP: Apoptosis and proliferation in lungs of ventilated and oxygen-treated preterm infants. Eur Respir J 2004, 23:113-121
18. Nagata S: Fas ligand-induced apoptosis. Annu Rev Genet 1999, 33:29-55
19. Sharma K, Wang R X, Zhang L Y, Yin D L, Luo X Y, Solomon J C, Jiang R F, Markos K, Davidson W, Scott D W, Shi Y F: Death the Fas way: regulation and pathophysiology of CD95 and its ligand. Pharmacol Ther 2000, 88:333-347
20. Walczak H, Krammer P H: The CD95 (APO-1/Fas) and the TRAIL (APO-2L) apoptosis systems. Exp Cell Res 2000, 256:58-66
21. Wajant H: The Fas signaling pathway: more than a paradigm. Science 2002, 296:1635-1636
22. De Paepe M E, Mao Q, Chao Y, Powell J L, Rubin L P, Sharma S: Hyperoxia-induced apoptosis and Fas/FasL expression in lung epithelial cells. Am J Physiol 2005, 289:L647-L659
23. Albertine K H, Soulier M F, Wang Z, Ishizaka A, Hashimoto S, Zimmerman G A, Matthay M A, Ware LB: Fas and fas ligand are up-regulated in pulmonary edema fluid and lung tissue of patients with acute lung injury and the acute respiratory distress syndrome. Am J Pathol 2002, 161:1783-1796
24. Hagimoto N, Kuwano K, Miyazaki H, Kunitake R, Fujita M, Kawasaki M, Kaneko Y, Hara N: Induction of apoptosis and pulmonary fibrosis in mice in response to ligation of Fas antigen. Am J Respir Cell Mol Biol 1997, 17:272-278
25. Hagimoto N, Kuwano K, Nomoto Y, Kunitake R, Hara N: Apoptosis and expression of Fas/Fas ligand mRNA in bleomycin-induced pulmonary fibrosis in mice. Am J Respir Cell Mol Biol 1997, 16:91-101
26. Kitamura Y, Hashimoto S, Mizuta N, Kobayashi A, Kooguchi K, Fujiwara I, Nakajima H: Fas/FasL-dependent apoptosis of alveolar cells after lipopolysaccharide-induced lung injury in mice. Am J Respir Crit Care Med 2001, 163:762-769
27. Kuwano K, Kawasaki M, Maeyama T, Hagimoto N, Nakamura N, Shirakawa K, Hara N: Soluble form of fas and fas ligand in BAL fluid from patients with pulmonary fibrosis and bronchiolitis obliterans organizing pneumonia. Chest 2000, 118:451-458
28. Kuwano K, Kunitake R, Maeyama T, Hagimoto N, Kawasaki M, Matsuba T, Yoshimi M, Inoshima I, Yoshida K, Hara N: Attenuation of bleomycin-induced pneumopathy in mice by a caspase inhibitor. Am J Physiol 2001, 280:L316-L325
29. Matute-Bello G, Liles W C, Frevert C W, Nakamura M, Ballman K, Vathanaprida C, Kiener P A, Martin T R: Recombinant human Fas ligand induces alveolar epithelial cell apoptosis and lung injury in rabbits. Am J Physiol 2001, 281:L328-L335
30. Matute-Bello G, Liles W C, Steinberg K P, Kiener P A, Mongovin S, Chi E Y, Jonas M, Martin T R: Soluble Fas ligand induces epithelial cell apoptosis in humans with acute lung injury (ARDS). J Immunol 1999, 163:2217-2225
31. Matute-Bello G, Frevert C W, Liles W C, Nakamura M, Ruzinski J T, Ballman K, Wong V A, Vathanaprida C, Martin T R: Fas/Fas ligand system mediates epithelial injury, but not pulmonary host defenses, in response to inhaled bacteria. Infect Immun 2001, 69:5768-5776
32. Nomoto Y, Kuwano K, Hagimoto N, Kunitake R, Kawasaki M, Hara N: Apoptosis and Fas/Fas ligand mRNA expression in acute immune complex alveolitis in mice. Eur Respir J 1997, 10:2351-2359
33. Perl M, Chung C S, Lomas-Neira J, Rachel T M, Biffl W L, Cioffi W G, Ayala A: Silencing of Fas, but not caspase-8, in lung epithelial cells ameliorates pulmonary apoptosis, inflammation, and neutrophil influx after hemorrhagic shock and sepsis. Am J Pathol 2005, 167:1545-1559
34. Gossen M, Freundlieb S, Bender G, Muller G, Hillen W, Bujard H: Transcriptional activation by tetracyclines in mammalian cells. Science 1995, 268:1766-1769
35. Kistner A, Gossen M, Zimmermann F, Jerecic J, Ullmer C, Lubbert H, Bujard H: Doxycycline-mediated quantitative and tissue-specific control of gene expression in transgenic mice. Proc Natl Acad Sci USA 1996, 93:10933-10938
36. Whitsett J A, Clark J C, Picard L, Tichelaar J W, Wert S E, Itoh N, Perl A K, Stahlman M T: Fibroblast growth factor 18 influences proximal programming during lung morphogenesis. J Biol Chem 2002, 277:22743-22749
37. Perl A K, Tichelaar J W, Whitsett J A: Conditional gene expression in the respiratory epithelium of the mouse. Transgenic Res 2002, 11:21-29
38. Tichelaar J W, Lu W, Whitsett J A: Conditional expression of fibroblast growth factor-7 in the developing and mature lung. J Biol Chem 2000, 275:11858-11864
39. Clark J C, Tichelaar J W, Wert S E, Itoh N, Perl A K, Stahlman M T, Whitsett J A: FGF-10 disrupts lung morphogenesis and causes pulmonary adenomas in vivo. Am J Physiol 2001, 280:L705-L715
40. Zhu Z, Ma B, Horner R J, Zheng T, Elias J A: Use of the tetracyclinecontrolled transcriptional silencer (tTS) to eliminate transgene leak in inducible overexpression transgenic mice. J Biol Chem 2001, 276:25222-25229
41. Zheng T, Zhu Z, Wang Z, Horner R J, Ma B, Riese Jr R J, Chapman Jr H A, Shapiro S D, Elias J A: Inducible targeting of IL-13 to the adult lung causes matrix metalloproteinase- and cathepsin-dependent emphysema. J Clin Invest 2000, 106:1081-1093
42. Yang L, Naltner A, Yan C: Overexpression of dominant negative retinoic acid receptor alpha causes alveolar abnormality in transgenic neonatal lungs. Endocrinology 2003, 144:3004-3011
43. Stripp B R, Sawaya P L, Luse D S, Wikenheiser K A, Wert S E, Huffman J A, Lattier D L, Singh G, Katyal S L, Whitsett J A: cis-Acting elements that confer lung epithelial cell expression of the CC10 gene. J Biol Chem 1992, 267:14703-14712
44. Takahashi T, Tanaka M, Brannan C I, Jenkins N A, Copeland N G, Suda T, Nagata S: Generalized lymphoproliferative disease in mice, caused by a point mutation in the Fas ligand. Cell 1994, 76:969-976
45. Amy R W, Bowes D, Burri P H, Haines J, Thurlbeck W M: Postnatal growth of the mouse lung. J Anat 1977, 124:131-151
46. Corti M, Brody A R, Harrison J H: Isolation and primary culture of murine alveolar type II cells. Am J Respir Cell Mol Biol 1996, 14:309-315
47. Rice W R, Conkright J J, Na C L, Ikegami M, Shannon J M, Weaver T E: Maintenance of the mouse type II cell phenotype in vitro. Am J Physiol 2002, 283:L256-L264
48. Dobbs L G: Isolation and culture of alveolar type II cells. Am J Physiol 1990, 258:L134-L147
49. Livak K J, Schmittgen T D: Analysis of relative gene expression data using real-time quantitative PCR and the 2(-Delta Delta C(T)) method. Methods 2001, 25:402-408
50. De Paepe M E, Johnson B D, Papadakis K, Luks F I: Lung growth response after tracheal occlusion in fetal rabbits is gestational agedependent. Am J Respir Cell Mol Biol 1999, 21:65-76
51. De Paepe M E, Johnson B D, Papadakis K, Sueishi K, Luks F I: Temporal pattern of accelerated lung growth after tracheal occlusion in the fetal rabbit. Am J Pathol 1998, 152:179-190
52. Aherne W A, Dunnill M S: The estimation of whole organ volume. Morphometry. Edited by Aherne W A, Dunnill M S. London, Edward Arnold Ltd., 1982, pp 10-18
53. De Paepe M E: Lung growth and development. Thurlbeck's Pathology of the Lung. Edited by Churg A M, Myers J L, Tazelaar H D, Wright J L. New York, Thieme Medical Publishers, 2005
54. Weibel E R, Cruz-Orive L M: Morphometric methods. The Lung: Scientific Foundations. Edited by Crystal R G, West J B, Weibel E R, Barnes P J. Philadelphia, Lippincott-Raven, 1997, pp 333-344
55. Balasubramaniam V, Mervis C F, Maxey A M, Markham N E, Abman S H: Hyperoxia reduces bone marrow, circulating, and lung endothelial progenitor cells in the developing lung: implications for the patho-genesis of bronchopulmonary dysplasia. Am J Physiol 2007, 292:L1073-L1084
56. Mantell L L, Shaffer T H, Horowitz S, Foust III R, Wolfson M R, Cox C, Khullar P, Zakeri Z, Lin L, Kazzaz J A, Palaia T, Scott W, Davis J M: Distinct patterns of apoptosis in the lung during liquid ventilation compared with gas ventilation. Am J Physiol 2002, 283:L31-L41
57. McGrath-Morrow S A, Stahl J: Apoptosis in neonatal murine lung exposed to hyperoxia. Am J Respir Cell Mol Biol 2001, 25:150-155
58. De Paepe M E, Mao Q, Luks F I: Expression of apoptosis-related genes after fetal tracheal occlusion in rabbits. J Pediatr Surg 2004, 39:1616-1625
59. Hammerschmidt S, Kuhn H, Grasenack T, Gessner C, Wirtz H: Apoptosis and necrosis induced by cyclic mechanical stretching in alveolar type II cells. Am J Respir Cell Mol Biol 2004, 30:396-402
60. Sanchez-Esteban J, Wang Y, Cicchiello L A, Rubin L P: Cyclic mechanical stretch inhibits cell proliferation and induces apoptosis in fetal rat lung fibroblasts. Am J Physiol 2002, 282:L448-L456
61. Edwards Y S, Sutherland L M, Power J H, Nicholas T E, Murray A W: Cyclic stretch induces both apoptosis and secretion in rat alveolar type II cells. FEBS Lett 1999, 448:127-130
62. Nakamura M, Matute-Bello G, Liles W C, Hayashi S, Kajikawa O, Lin S M, Frevert C W, Martin T R: Differential response of human lung epithelial cells to fas-induced apoptosis. Am J Pathol 2004, 164:1949-1958
63. Mason R J, Shannon J M: Alveolar type II cells. The Lung. Edited by Crystal R G, West J B. Philadelphia, Lippincott-Raven, 1997, pp 543-555
64. Kasahara Y, Tuder R M, Taraseviciene-Stewart L, Le Cras T D, Abman S, Hirth P K, Waltenberger J, Voelkel N F: Inhibition of VEGF receptors causes lung cell apoptosis and emphysema. J Clin Invest 2000, 106:1311-1319
65. Tuder R M, Zhen L, Cho C Y, Taraseviciene-Stewart L, Kasahara Y, Salvemini D, Voelkel N F, Flores S C: Oxidative stress and apoptosis interact and cause emphysema due to vascular endothelial growth factor receptor blockade. Am J Respir Cell Mol Biol 2003, 29:88-97
66. Lucey E C, Keane J, Kuang P P, Snider G L, Goldstein R H: Severity of elastase-induced emphysema is decreased in tumor necrosis factoralpha and interleukin-1beta receptor-deficient mice. Lab Invest 2002, 82:79-85
67. Imai K, Dalal S S, Chen E S, Downey R, Schulman L L, Ginsburg M, D′Armiento J: Human collagenase (matrix metalloproteinase-1) expression in the lungs of patients with emphysema. Am J Respir Crit Care Med 2001, 163:786-791
68. Kasahara Y, Tuder R M, Cool C D, Lynch D A, Flores S C, Voelkel N F: Endothelial cell death and decreased expression of vascular endothelial growth factor and vascular endothelial growth factor receptor 2 in emphysema. Am J Respir Crit Care Med 2001, 163:737-744
69. Lee C G, Kang H R, Horner R J, Chupp G, Elias J A: Transgenic modeling of transforming growth factor-beta(1): role of apoptosis in fibrosis and alveolar remodeling. Proc Am Thorac Soc 2006, 3:418-423
70. Aoshiba K, Yokohori N, Nagai A: Alveolar wall apoptosis causes lung destruction and emphysematous changes. Am J Respir Cell Mol Biol 2003, 28:555-562
71. Speer C P: Inflammation and bronchopulmonary dysplasia: a continuing story. Semin Fetal Neonatal Med 2006, 11:354-362
72. Kallapur S G, Jobe A H: Contribution of inflammation to lung injury and development. Arch Dis Child Fetal Neonatal Ed 2006, 91:F132-F135
73. Thebaud B, Abman S H: Bronchopulmonary dysplasia: where have all the vessels gone? Roles of angiogenic growth factors in chronic lung disease. Am J Respir Crit Care Med 2007, 175:978-985
74. Bhatt A J, Pryhuber G S, Huyck H, Watkins R H, Metlay L A, Maniscalco W M: Disrupted pulmonary vasculature and decreased vascular endothelial growth factor. Flt-1, and TIE-2 in human infants dying with bronchopulmonary dysplasia. Am J Respir Crit Care Med 2001, 164:1971-1980
75. Coalson J J, Winter V T, Siler-Khodr T, Yoder B A: Neonatal chronic lung disease in extremely immature baboons. Am J Respir Crit Care Med 1999, 160:1333-1346
76. Maniscalco W M, Watkins R H, Pryhuber G S, Bhatt A, Shea C, Huyck H: Angiogenic factors and alveolar vasculature: development and alterations by injury in very premature baboons. Am J Physiol 2002, 282:L811-L823
77. Gochuico B R, Miranda K M, Hessel E M, De Bie J J, Van Oosterhout A J, Cruikshank W W, Fine A: Airway epithelial Fas ligand expression: potential role in modulating bronchial inflammation. Am J Physiol 1998, 274:L444-L449
78. Hamann K J, Dorscheid D R, Ko F D, Conforti A E, Sperling A I, Rabe K F, White S R: Expression of Fas (CD95) and FasL (CD95L) in human airway epithelium. Am J Respir Cell Mol Biol 1998, 19:537-542
79. Martin T R, Hagimoto N, Nakamura M, Matute-Bello G: Apoptosis and epithelial injury in the lungs. Proc Am Thorac Soc 2005, 2:214-220
80. You M, Ku P T, Hrdlickova R, Bose Jr R H R: ch-IAP 1, a member of the inhibitor-of-apoptosis protein family, is a mediator of the antiapoptotic activity of the v-Rel oncoprotein. Mol Cell Biol 1997, 17:7328-7341
81. Hagimoto N, Kuwano K, Kawasaki M, Yoshimi M, Kaneko Y, Kunitake R, Maeyama T, Tanaka T, Hara N: Induction of interleukin-8 secretion and apoptosis in bronchiolar epithelial cells by Fas ligation. Am J Respir Cell Mol Biol 1999, 21:436-445
82. Whitsett J A, Perl A K: Conditional control of gene expression in the respiratory epithelium: a cautionary note. Am J Respir Cell Mol Biol 2006, 34:519-520
83. Hosford G E, Fang X, Olson D M: Hyperoxia decreases matrix metalloproteinase-9 and increases tissue inhibitor of matrix metalloproteinase-1 protein in the newborn rat lung: association with arrested alveolarization. Pediatr Res 2004, 56:26-34
84. Sorsa T, Ding Y, Salo T, Lauhio A, Teronen O, Ingman T, Ohtani H, Andoh N, Takeha S, Konttinen Y T: Effects of tetracyclines on neutrophil, gingival, and salivary collagenases. A functional and Westernblot assessment with special reference to their cellular sources in periodontal diseases. Ann NY Acad Sci 1994, 732:112-131
85. Sapadin A N, Fleischmajer R: Tetracyclines: nonantibiotic properties and their clinical implications. J Am Acad Dermatol 2006, 54:258-265 Transgenic FasL Mouse 15 AJP July 2008, Vol. 173, No. 1

The teachings of all patents, published applications and references cited herein are incorporated by reference in their entirety.

### EQUIVALENTS

While this invention has been particularly shown and described with references to example embodiments thereof, it will be understood by those skilled in the art that various changes in form and details may be made therein without departing from the scope of the invention encompassed by the appended claims.

What is claimed is:

1. A transgenic non-human animal whose genome comprises a stable integration of at least one transgene that includes at least one nucleic acid sequence encoding at least one Fas-ligand protein operably-linked to at least one tetracycline-inducible promoter, wherein at least one cell of the transgenic non-human animal that expresses the transgene undergoes apoptosis and wherein the non-human animal is not a rat.

2. The transgenic non-human animal of claim 1, wherein the cell is a somatic cell.

3. The transgenic non-human animal of claim 1, wherein the cell is a germ cell.

4. The transgenic non-human animal of claim 1, wherein the animal is fertile.

5. The transgenic non-human animal of claim 1, wherein the animal survives when apoptosis is induced.

6. The transgenic non-human animal of claim 1, wherein the transgenic non-human animal is a mouse.

7. The transgenic non-human animal of claim 1, wherein the genome of the transgenic non-human animal includes the stable integration of about two to about thirty-five copies of the transgene.

8. The transgenic non-human animal of claim 7, wherein the genome of the transgenic non-human animal includes the stable integration of about twenty copies of the transgene.

9. The transgenic non-human animal of claim 8, wherein the cell of the transgenic non-human animal that expresses the transgene has about a 10-fold to about a 200-fold increase in Fas-ligand mRNA levels compared to a control cell.

10. The transgenic non-human animal of claim 9, wherein the cell of the transgenic non-human animal that expresses the transgene has at least about a 30-fold increase in Fas-ligand mRNA levels compared to a control cell.

11. The transgenic non-human animal of claim 1, wherein the nucleic acid sequence encoding the Fas-ligand protein has at least about 75% identity to SEQ ID NO:4.

12. The transgenic non-human animal of claim 1, wherein the tetracycline-inducible promoter includes at least about seven copies of a tet operator nucleic acid sequence.

13. The transgenic non-human animal of claim 12, wherein the tetracycline-inducible promoter includes seven copies of the tet operator nucleic acid sequence.

14. The transgenic non-human animal of claim 1, wherein the tetracycline-inducible promoter includes a cytomegalovirus minimal promoter nucleic acid sequence.

15. The transgenic non-human animal of claim 1, wherein the transgene further includes a polyadenylation nucleic acid sequence.

16. A transgenic non-human animal whose genome comprises a stable integration of at least one first transgene that includes at least one nucleic acid sequence encoding at least one Fas-ligand protein operably-linked to at least one tetracycline-inducible promoter and at least one second transgene that includes at least one second nucleic acid sequence encoding at least one member selected from the group consisting of a reverse tetracycline responsive transactivator protein and a tetracycline responsive transactivator protein.

17. The transgenic non-human animal of claim 16, wherein at least one cell of the transgenic non-human animal that co-expresses both the first transgene and the second transgene undergoes apoptosis.

18. The transgenic non-human animal of claim 17, wherein the second transgene includes at least one second nucleic acid sequence encoding a reverse tetracycline responsive transactivator protein.

19. The transgenic non-human animal of claim 18, wherein apoptosis is induced by at least one member selected from the group consisting of a tetracycline and a tetracycline analog.

20. The transgenic non-human animal of claim 17, wherein the second transgene includes at least one member selected from the group consisting of a pancreatic β-cell promoter, an amyloid precursor protein gene promoter, a dystrophin gene promoter, a Clara cell secretory protein gene promoter, a surfactant protein-B gene promoter, a surfactant protein-C gene promoter, an insulin gene promoter, an albumin gene promoter, an alpha Calcium/Calmodulin dependent Protein Kinase II gene promoter, a neuron-specific enolase gene promoter, a retinoblastoma gene promoter, a muscle creatine kinase gene promoter, an alpha myosin heavy chain gene promoter, a TEK tyrosine kinase gene promoter, a Tie receptor tyrosine kinase gene promoter, an immunoglobulin heavy chain enhancer, a CD34 gene promoter, an SM22alpha gene promoter, and a glial fibrillary acidic gene promoter.

21. The transgenic non-human animal of claim 17, wherein the cell is an epithelial tissue cell.

22. The transgenic non-human animal of claim 21, wherein the epithelial tissue cell is a lung epithelial cell.

23. The transgenic non-human animal of claim 22, wherein the lung epithelial cell is a ciliated lung epithelial cell.

24. The transgenic non-human animal of claim 22, wherein the lung epithelial cell is an alveolar lung epithelial cell.

25. The transgenic non-human animal of claim 24, wherein the alveolar lung epithelial cell is a type II alveolar lung epithelial cell.

26. The transgenic non-human animal of claim 22, wherein the lung epithelial cell is a nonciliated lung epithelial cell.

27. The transgenic non-human animal of claim 26, wherein the nonciliated lung epithelial cell is a nonciliated bronchial epithelial cell.

28. The transgenic non-human animal of claim 20, wherein the second transgene includes the Clara cell secretory protein gene promoter.

29. The transgenic non-human animal of claim 28, wherein the animal has a phenotype of at least one member selected from the group consisting of an alveolar type II cell apoptosis, a nonciliated bronchial epithelial cell apoptosis, a disrupted alveolar development, a decreased vascular density and an increased postnatal lethality consequent to apoptosis.

30. The transgenic non-human animal of claim 29, wherein the disrupted alveolar development includes alveolar simplification that resembles pulmonary pathology of human bronchopulmonary dysplasia.

31. The transgenic non-human animal of claim 16, wherein the nucleic acid sequence encoding the reverse tetracycline responsive transactivator protein or the nucleic acid sequence encoding the tetracycline responsive transactivator protein is operably linked to at least one promoter selected from the group consisting of a cell-specific promoter and a tissue-specific promoter.

32. The transgenic non-human animal of claim 18, wherein the second nucleic acid sequence encoding a reverse tetracycline responsive transactivator protein is operably linked to at least one promoter that includes a rat Clara cell secretory protein gene promoter element.

33. A recombinant nucleic acid comprising a nucleotide sequence having at least about 75% identity to SEQ ID NO:4 operably-linked to a tetracycline-inducible promoter, wherein the tetracycline-inducible promoter includes at least seven copies of a tetracycline operator nucleic acid sequence and a cytomegalovirus minimal promoter nucleic acid sequence.

34. A method for producing a transgenic non-human animal, comprising the step of crossing a first transgenic non-human animal whose genome comprises a stable integration of at least one first transgene that includes at least one nucleic acid sequence encoding at least one Fas-ligand protein operably-linked to a tetracycline-inducible promoter with a second transgenic non-human animal whose genome comprises a stable integration of at least one second transgene that includes at least one second nucleic acid sequence encoding at least one reverse tetracycline responsive transactivator protein.

35. The method of claim 34, wherein at least one cell of the transgenic non-human animal that co-expresses both the first transgene and the second transgene undergoes apoptosis.

36. The method of claim 34, wherein the first transgenic non-human animal and second transgenic non-human animal are mice.

37. The method of claim 36, wherein the mice have an FVB/N genetic background.

38. A method of screening for a compound that inhibits Fas-ligand mediated apoptosis, comprising the step of assessing Fas-ligand mediated apoptosis in a transgenic non-human animal whose genome comprises a stable integration of at least one first transgene that includes at least one nucleic acid sequence encoding at least one Fas-ligand protein operably-linked to at least one tetracycline-inducible promoter and at least one second transgene that includes at least one second nucleic acid sequence encoding at least one reverse tetracycline responsive transactivator protein, wherein the Fas-ligand mediated apoptosis is in response to administration of the compound in combination with at least one member selected from the group consisting of a tetracycline and a tetracycline analog to the transgenic non-human animal.

39. A method of identifying a cell that is capable of differentiating into a target cell, comprising the steps of:
(a) inducing apoptosis of a population of target cells in a transgenic non-human animal whose genome comprises a stable integration of at least one first transgene that includes at least one nucleic acid sequence encoding at least one Fas-ligand protein operably-linked to at least one tetracycline-inducible promoter and at least one second transgene that includes a second nucleic acid sequence encoding at least one member selected from the group consisting of a reverse tetracycline responsive transactivator protein and a tetracycline responsive transactivator protein, and wherein the first transgene and the second transgene are co-expressed in the target cells;
(b) introducing at least one cell into the transgenic non-human animal, wherein the cell is selected form the group consisting of a stem cell, a progenitor cell and a bone marrow-derived cell; and
(c) detecting differentiation of the cell into a phenotype characteristic of the target cell.


SEQUENCE LISTING (13 sequences)

SEQ ID NO: 1 - protein, 278 aa, Mus musculus
MQQPMNYPCPQIFWVDSSATSSWAPPGSVFPCPSCGPRGPDQRRPPPPPPPVSPLPPPSQPLPLPPLTPLKKKDHNTNLWLPVVFFMVLVALVGMGLGMYQLFHLQKELAELREFTNQSLKVSSFEKQIANPSTPSEKKEPRSVAHLTGNPHSRSIPLEWEDTYGTALISGVKYKKGGLVINETGLYFVYSKVYFRGQSCNNQPLNHKVYRNSKYPEDLVLMEEKRLNYCTTGQIWAHSSYLGAVFNLTSADHLYVNISQLSLINFEESKTFFGLYKL

SEQ ID NO: 2 - protein, 278 aa, Rattus norvegicus
MQQPVNYPCPQIYWVDSSATSPWAPPGSVFSCPSSGPRGPGQRRPPPPPPPPSPLPPPSQPPPLPPLSPLKKKDNIELWLPVIFFMVLVALVGMGLGMYQLFHLQKELAELREFTNHSLRVSSFEKQIANPSTPSETKKPRSVAHLTGNPRSRSIPLEWEDTYGTALISGVKYKKGGLVINEAGLYFVYSKVYFRGQSCNSQPLSHKVYMRNFKYPGDLVLMEEKKLNYCTTGQIWAHSSYLGAVFNLTVADHLYVNISQLSLINFEESKTFFGLYKL

SEQ ID NO: 3 - protein, 281 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 4 - DNA, 1937 bp, Mus musculus
tgaggcttctcagcttcagatgcaagtgagtgggtgtctcacagagaagcaaagagaagagaacaggagaaaggtgtttcccttgactgcggaaactttataaagaaaacttagcttctctggagcagtcagcgtcagagttctgtccttgacacctgagtctcctccacaaggctgtgagaaggaaaccctttcctggggctgggtgccatgcagcagcccatgaattacccatgtccccagatcttctgggtagacagcagtgccacttcatcttgggctcctccagggtcagtttttccctgtccatcttgtgggcctagagggccggaccaaaggagaccgccacctccaccaccacctgtgtcaccactaccaccgccatcacaaccactcccactgccgccactgacccctctaaagaagaaggaccacaacacaaatctgtggctaccggtggtatttttcatggttctggtggctctggttggaatgggattaggaatgtatcagctcttccacctgcagaaggaactggcagaactccgtgagttcaccaaccaaagccttaaagtatcatcttttgaaaagcaaatagccaaccccagtacaccctctgaaaaaaaagagccgaggagtgtggcccatttaacagggaacccccactcaaggtccatccctctggaatgggaagacacatatggaaccgctctgatctctggagtgaagtataagaaaggtggccttgtgatcaacgaaactgggttgtacttcgtgtattccaaagtatacttccggggtcagtcttgcaacaaccagcccctaaaccacaaggtctatatgaggaactctaagtatcctgaggatctggtgctaatggaggagaagaggttgaactactgcactactggacagatatgggcccacagcagctacctgggggcagtattcaatcttaccagtgctgaccatttatatgtcaacatatctcaactctctctgatcaattttgaggaatctaagacctttttcggcttgtataagctttaaaagaaaaagcattttaaaatgatctactattctttatcatgggcaccaggaatattgtcttgaatgagagtcttcttaagacctattgagattaattaagactacatgagccacaaagacctcatgaccgcaaggtccaacaggtcagctatccttcattttctcgaggtccatggagtggtccttaatgcctgcatcatgagccagatggaaggaggtctgtgactgagggacataaagctttgggctgctgtgtgacaatgcagaggcacagagaaagaactgtctgatgttaaatggccaagagaattttaaccattgaagaagacacctttacactcacttccagggtgggtctacttactacctcacagaggccgtttttgagacatagttgtggtatgaatatacaagggtgagaaaggaggctcatttgactgataagctagagactgaaaaaaagacagtgtctcattggcaccatctttactgttacctaatgttttctgagccgacctttgatcctaacggagaagtaagagggatgtttgaggcacaaatcattctctacatagcatgcatacctccagtgcaatgatgtctgtgtgtttgtatgtatgagagcaaacagattctaaggagtcatataaataaaatatgtacattatggagtacatattagaaacctgttacatttgatgctagatatctgaatgtttcttggcaataaactctaatagtcttcaaaatcttttattatcagctactgatgctgtttttctttaatacaactagtatttatgctctgaacatcctaatgaggaaaagacaaataaaattatgttatagaatacagaaatgccttaaggacatagactttggaaatc

SEQ ID NO: 5 - DNA, 1623 bp, Rattus norvegicus
tcagagtcctgtccttgacacttcagtctccacaagactgagaggaggaaaccctttcctggggctgggtgccatgcagcagcccgtgaattacccatgtccccagatctactgggtagacagcagtgccacttctccttgggctcctccagggtcagttttttcttgtccatcctctgggcctagagggccaggacaaaggagaccaccgcctccaccaccacctccatcaccactaccaccgccttcccaaccacccccgctgcctccactaagccctctaaagaagaaggacaacatagagctgtggctaccggtgatatttttcatggtgctggtggctctggttggaatggggttaggaatgtatcaactctttcatctacagaaggaactggcagaactccgtgagttcaccaaccacagccttagagtatcatcttttgaaaagcaaatagccaaccccagcacaccctctgaaaccaaaaagccaaggagtgtggcccacttaacagggaacccccgctcaaggtccatccctctggaatgggaagacacatatggaactgctttgatctctggagtgaagtataagaaaggcggccttgtgatcaatgaggctgggttgtacttcgtatattccaaagtatacttccggggtcagtcttgcaacagccagcccctaagccacaaggtctatatgaggaactttaagtatcctggggatctggtgctaatggaggagaagaagttgaattactgcactactggccagatatgggcccacagcagctacctaggggcagtatttaatcttaccgttgctgaccatttatatgtcaacatatctcaactctctctgatcaattttgaggaatctaagaccttttttggcttatataagctttaaaggaaaaagcattttagaatgatctattattctttatcatggatgccaggaatattgtcttcaatgagagtcttcttaagaccaattgagccacaaagaccacaaggtccaacaggtcagctacccttcattttctagaggtccatggagtggtccttaatgcctgcatcatgagccagatgggaagaagactgttcctgaggaacataaagttttgggctgctgtgtggcaatgcagaggcaaagagaaggaactgtctgatgttaaatggccaagagcattttagccattgaagaaaaaaaaaacctttaaactcaccttccagggtgggtctacttgctacctcacaggaggccgtcttttagacacatggttgtggtatgactatacaagggtgagaaaggatgctaggtttcatggataagctagagactgaaaaaagccagtgtcccattggcatcatctttatttttaactgatgttttctgagcccacctttgatgctaacagagaaataagaggggtgtttgaggcacaagtcattctctacatagcatgtgtacctccagtgcaatgatgtctgtgtgtgtttttatgtatgagagtagagcgattctaaagagtcacatgagtacaacgcgtacattacggagtacatattagaaacgtatgtgttacatttgatgctagaatatctgaatgtttcttgcta

SEQ ID NO: 6 - DNA, 1909 bp, Homo sapiens
gaggtgtttcccttagctatggaaactctataagagagatccagcttgcctcctcttgagcagtcagcaacagggtcccgtccttgacacctcagcctctacaggactgagaagaagtaaaaccgtttgctggggctggcctgactcaccagctgccatgcagcagcccttcaattacccatatccccagatctactgggtggacagcagtgccagctctccctgggcccctccaggcacagttcttccctgtccaacctctgtgcccagaaggcctggtcaaaggaggccaccaccaccaccgccaccgccaccactaccacctccgccgccgccgccaccactgcctccactaccgctgccacccctgaagaagagagggaaccacagcacaggcctgtgtctccttgtgatgtttttcatggttctggttgccttggtaggattgggcctggggatgtttcagctcttccacctacagaaggagctggcagaactccgagagtctaccagccagatgcacacagcatcatctttggagaagcaaataggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctctaagagaagcactttgggattctttccattatgattctttgttacaggcaccgagaatgttgtattcagtgagggtcttcttacatgcatttgaggtcaagtaagaagacatgaaccaagtggaccttgagaccacagggttcaaaatgtctgtagctcctcaactcacctaatgtttatgagccagacaaatggaggaatatgacggaagaacatagaactctgggctgccatgtgaagagggagaagcatgaaaaagcagctaccaggtgttctacactcatcttagtgcctgagagtatttaggcagattgaaaaggacaccttttaactcacctctcaaggtgggccttgctacctcaagggggactgtctttcagatacatggttgtgacctgaggatttaagggatggaaaaggaagactagaggcttgcataataagctaaagaggctgaaagaggccaatgccccactggcagcatcttcacttctaaatgcatatcctgagccatcggtgaaactaacagataagcaagagagatgttttggggactcatttcattcctaacacagcatgtgtatttccagtgcaattgtaggggtgtgtgtgtgtgtgtgtgtgtgtgtgtgtgtatgactaaagagagaatgtagatattgtgaagtacatattaggaaaatatgggttgcatttggtcaagattttgaatgcttcctgacaatcaactctaatagtgcttaaaaatcattgattgtcagctactaatgatgttttcctataatataataaatatttatgtagatgtgcatttttgtgaaatgaaaacatgtaataaaaagtatatgttaggatacaaaaaaaaaaaaaaaaaaaaaaaaaaaaaaaaaaaaaaaaaaaaaaaaaaaa

SEQ ID NO: 7 - DNA, 438 bp, Artificial Sequence
  tet-responsive promoter
ctcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgaggtaggcgtgtacggtgggaggcctatataagcagagctcgtttagtgaaccgtcagatcgcctggagacgccatccacgctgttttgacctccatagaagacaccgggaccgatccagcc

SEQ ID NO: 8 - DNA, 119 bp, Cytomegalovirus
taggcgtgtacggtgggaggcctatataagcagagctcgtttagtgaaccgtcagatcgcctggagacgccatccacgctgttttgacctccatagaagacaccgggaccgatccagcc

SEQ ID NO: 9 - DNA, 1168 bp, Unknown
  beta-globin polyadenylation signal
agctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaacttttttcgttaaactttagcttgcatttgtaacgaatttttaaattcactttcgtttatttgtcagattgtaagtactttctctaatcacttttttttcaaggcaatcagggtaattatattgtacttcagcacagttttagagaacaattgttataattaaatgataaggtagaatatttctgcatataaattctggctggcgtggaaatattcttattggtagaaacaactacatcctggtaatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaagaattcactcctcaggtgcaggctgcctatcagaaggtggtggctggtgtggccaatgccctggctcacaaataccactgagatctttttccctctgccaaaaattatggggacatcatgaagccccttgagcatctgacttctgggtaataaaggaaatttattttcattgcaatagtgtgtgggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaactcgact

SEQ ID NO: 10 - DNA, 941 bp, Mus musculus
tctagagagaaggaaaccctttcctggggctgggtgccatgcagcagcccatgaattacccatgtccccagatcttctgggtagacagcagtgccacttcatcttgggctcctccagggtcagtttttccctgtccatcttgtgggcctagagggccggaccaaaggagaccgccacctccaccaccacctgtgtcaccactaccaccgccatcacaaccactcccactgccgccactgacccctctaaagaagaaggaccacaacacaaatctgtggctaccggtggtatttttcatggttctggtggctctggttggaatgggattaggaatgtatcagctcttccacctgcagaaggaactggcagaactccgtgagttcaccaaccaaagccttaaagtatcatcttttgaaaagcaaatagccaaccccagtacaccctctgaaaaaaaagagccgaggagtgtggcccatttaacagggaacccccactcaaggtccatccctctggaatgggaagacacatatggaaccgctctgatctctggagtgaagtataagaaaggtggccttgtgatcaacgaaactgggttgtacttcgtgtattccaaagtatacttccggggtcagtcttgcaacaaccagcccctaaaccacaaggtctatatgaggaactctaagtatcctgaggatctggtgctaatggaggagaagaggttgaactactgcactactggacagatatgggcccacagcagctacctgggggcagtattcaatcttaccagtgctgaccatttatatgtcaacatatctcaactctctctgatcaattttgaggaatctaagacctttttcggcttgtataagctttaaaagaaaaagcattttaaaatgatctactattctttatcatgggcaccaggaatattctagagc

SEQ ID NO: 11 - DNA, 17 bp, Artificial Sequence
  Oligonucleotide primer
cgcctggagacgccatc

SEQ ID NO: 12 - DNA, 21 bp, Artificial Sequence
  Oligonucleotide primer
gtgccatgcagcagcccatga

SEQ ID NO: 13 - DNA, 20 bp, Artificial Sequence
  Oligonucleotide primer
ccattctaaacaacaccctg